(19) United States
(12) Patent Application Publication
(10) Pub. No.: US 2024/0245779 A1
(43) Pub. Date: Jul. 25, 2024

(54) METHODS OF MODELING LIQUID PROTEIN COMPOSITION STABILITY

(71) Applicant: Regeneron Pharmaceuticals, Inc., Tarrytown, NY (US)

(72) Inventors: Douglas E. KAMEN, Poughquag, NY (US); Kenneth S. GRAHAM, Tarrytown, NY (US); Caterina RICCARDI, Tarrytown, NY (US)

(21) Appl. No.: 18/420,955

(22) Filed: Jan. 24, 2024

Related U.S. Application Data
(60) Provisional application No. 63/481,443, filed on Jan. 25, 2023.

(51) Int. Cl. A61K 47/26; A61J 1/10; A61K 39/395; A61K 47/68; G01N 33/15

(52) U.S. Cl. CPC A61K 47/26; A61K 39/395; A61K 47/68; G01N 33/15; A61J 1/10

(57) ABSTRACT

The disclosure provides methods of determining an initial amount of surfactant to include in a liquid pharmaceutical composition comprising a protein, intended for administration to a subject as an IV admixture. The methods comprise determining the degradation rate of the surfactant, the minimum amount of surfactant whereby stability of the protein is maintained in IV admixture the end of shelf-life of the liquid pharmaceutical composition, and, based on the degradation rate, shelf-life, and minimum amount of surfactant, determining a target amount of surfactant to include in the composition at the time of formulation.

### CROSS REFERENCE TO RELATED APPLICATION

This application claims priority to, and benefit of, U.S. Provisional Application No. 63/481,443, filed on Jan. 25, 2023, the contents of which are incorporated by reference in their entirety herein.

### BACKGROUND

Polysorbate 20 and 80 are widely used surfactants in liquid biopharmaceutical protein formulations. However, polysorbates are known to degrade over time. Polysorbate degradation can lead to protein instability resulting in the formation of sparingly soluble compounds such as free fatty acid (FFA) particles, reducing the shelf-life of the biopharmaceutical formulation, and leading to unacceptably high levels of particulates. There thus exists a need in the art to better understand the effects of polysorbate degradation on liquid biopharmaceutical formulations, particularly when a drug product is stored and diluted to produce an intravenous (IV) admixture.

### SUMMARY

The disclosure provides methods of determining a target amount of surfactant in a liquid pharmaceutical composition comprising a protein.

In some embodiments of the methods of the disclosure, the methods comprise determining a target amount of surfactant in a liquid pharmaceutical composition comprising a protein, whereby stability of the protein is maintained in an IV admixture comprising the liquid pharmaceutical composition, comprising: (a) generating a plurality of liquid pharmaceutical compositions, wherein liquid pharmaceutical compositions in the plurality differ by an amount of the surfactant present in the liquid pharmaceutical compositions; (b) generating a plurality of IV admixtures from the plurality of liquid pharmaceutical compositions by mixing each liquid pharmaceutical composition with a diluent suitable for intravenous (IV) administration in a container; (c) simulating intravenous delivery of the plurality of IV admixtures to a subject; (d) measuring particles per container of IV admixture for IV admixtures in the plurality; (e) determining a minimum amount of surfactant whereby an amount of particles per container of IV admixture does not exceed more than 6000 particles greater than 10 μm and 600 particles greater than 25 μm; and (f) based on a shelf-life of the liquid pharmaceutical composition, the minimum amount of surfactant from step (c), and a degradation rate of the surfactant, determining the target amount of surfactant in the liquid pharmaceutical composition whereby stability of the protein is maintained in the IV admixture when the IV admixture is formulated at the end of the shelf-life of the liquid pharmaceutical composition. In some embodiments, the methods comprise determining the degradation rate of the surfactant by: (i) determining an initial amount of surfactant in the liquid pharmaceutical composition; (ii) holding the liquid pharmaceutical composition for at least a first amount of time; (iii) determining at least a second amount of surfactant in the liquid pharmaceutical composition; and (iv) applying a model of surfactant concentration over time. In some embodiments, simulating intravenous delivery of the plurality of IV admixtures to a subject comprises (i) incubating the plurality IV admixtures for a first period of time at 2-8° C.; (ii) incubating for a second period of time at 21-26° C.; and (iii) pumping the plurality IV admixtures into receptacles.

The disclosure provides liquid pharmaceutical compositions comprising an amount surfactant determined by the methods of the disclosure, wherein the liquid pharmaceutical composition is suitable for use in an IV admixture.

The disclosure provides methods of determining a maximum amount of time a liquid pharmaceutical composition comprising a protein and a surfactant can be stored (shelf-life).

In some embodiments of the methods of the disclosure, the methods comprise (a) generating a plurality of liquid pharmaceutical compositions, wherein liquid pharmaceutical compositions in the plurality differ by an amount of the surfactant present in the liquid pharmaceutical compositions; (b) generating a plurality of IV admixtures from the plurality of liquid pharmaceutical compositions by mixing each liquid pharmaceutical composition with a diluent suitable for intravenous (IV) administration in a container; (c) simulating intravenous delivery of the plurality of IV admixtures to a subject; (d) measuring particles per container of IV admixture for IV admixtures in the plurality; (c) determining a minimum amount of surfactant whereby an amount of particles per container of IV admixture does not exceed more than 6000 particles greater than 10 μm and 600 particles greater than 25 μm; and (f) based on a rate of degradation of the surfactant, the minimum amount of surfactant from step (c), and an initial amount of surfactant in the liquid pharmaceutical composition, determining the maximum shelf-life of the liquid pharmaceutical composition whereby stability of the protein is maintained in the IV admixture when the IV admixture is formulated at the end of the shelf-life of the liquid pharmaceutical composition. In some embodiments, the methods comprise determining the degradation rate of e surfactant by: (i) determining an initial amount of surfactant in the liquid pharmaceutical composition; (ii) holding the liquid pharmaceutical composition for at least a first amount of time; (iii) determining at least a second amount of surfactant in the liquid pharmaceutical composition; and (iv) applying a model of surfactant concentration over time. In some embodiments, simulating intravenous delivery of the plurality of IV admixtures to a subject comprises (i) incubating the plurality IV admixtures for a first period of time at 2-8° C.; (ii) incubating for a second period of time at 21-26° C.; and (iii) pumping the plurality IV admixtures into receptacles.

In some embodiments, the surfactant comprises a polysorbate. In some embodiments, the polysorbate comprises polysorbate 20.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

FIG. 1A is a plot showing particle analysis by flow imaging (FI) for high and low antibody concentration drug product (DP) formulations of monoclonal antibody 1 (mAb1). Spikes in particle formation are observed after 18 months of storage at 5° C. and continue to be elevated for the duration of the study.

FIG. 1B is a plot showing particle analysis by light obscuration (LO) for high and low antibody concentration drug product (DP) formulations of monoclonal antibody 1 (mAb1). The plot shows no meaningful increases in particle counts over the course of the study.

FIG. 1C is a plot showing relative percentages of Polysorbate 20 levels in mAb1 high and low concentration DP vials. The y-axis, polysorbate concentration in high mAb1 concentration (circles) and low mAb1 concentration (diamonds) formulations. Polysorbate concentration is reported as percent weight/volume (w/v) of the formulation.

FIG. 1D is a plot showing particle analysis by FI for high (200 mg/mL) and low (60 mg/mL) DP presentations of mAb1. Increases in particle formation are observed after 18 months of storage at 5° C. and continue to be elevated for the duration of the study.

FIG. 1E is a plot showing particle analysis by LO for high (200 mg/mL) and low (60 mg/mL) DP presentations of mAb1, and shows no meaningful increases in particle counts over the course of the study with the exception of an increase in 2-10 μm particle counts. Reference lines indicating the USP <788> limits are shown.

FIG. 1F is a plot showing polysorbate levels in mAB1 200 mg/mL and 60 mg/mL DP vials. Data were fit to a single exponential decay model shown in Equation 1.

FIG. 2A is a series of FI images of particles formed in a mAb1 DP. The particles shift to more fibrous particles during storage (in months, or “m”) at 5° C. ECD: equivalent circular diameter.

FIG. 2B is a plot showing Raman microscopy of a particle from mAb1 DP at 18 months of storage at 5° C. The raw spectrum of the particle is shown in red, the sample spectrum after mathematical smoothing and correction is shown in blue, and the closest database hit (myristic acid match rank 966 out of 1000) is shown in green. The second closest Raman match was lauric acid (match rank 948 out of 1000, spectra not shown).

FIG. 2C is a plot showing Raman microscopy of a particle from mAb1 DP at 18 months of storage at 5° ° C. The raw spectrum of the particle is shown in red at bottom, and the closest database match to myristic acid (myristic acid match rank 996 out of 1000) is shown in green, at top.

FIG. 3A is a series of particle images after storage of admixtures when polysorbate is not included in the DP formulation, as determined by FI.

FIG. 3B is a plot showing particle levels in IV admixtures determined by LO for admixture solutions containing 0% (w/v) polysorbate 20. USP <788> limits are indicated. 100 mL IV bag sizes were tested.

FIG. 3C is a plot showing particle levels in IV admixtures, as determined by LO. Particle size is indicated on the y-axis, with the lines marked USP <788> indicating the acceptable levels of particles indicated in Chapter <788> of the United States Pharmacopeia (USP) Particulate Matter in Injections, i.e., that particles do not exceed 6000 particles ≥10 μm and 600 particles ≥25 μm per container. The percent polysorbate 20 in the IV admixture is indicated on the x-axis. Circles indicate measurements from 50 mL IV bags, while triangles indicate measurements from 100 mL IV bags. The percent polysorbate (PS) 20 in the drug product relative to the target polysorbate level is shown in the color key at top right.

FIG. 3D is a plot showing particle levels in IV admixtures determined by LO. The samples are from the study described in FIGS. 4A-4B. USP <788> limits are indicated. The IV bag sizes tested are shown by the circles (50 mL) or triangles (100 mL). the solid symbols include the t=0 and storage samples, while the open symbols show the samples delivered through the IV apparatus. The initial polysorbate 20 levels in the vial DP are indicated by the key at top right.

FIG. 4A is a diagram showing the design of a study to simulate polysorbate degradation in IV admixtures. DP with polysorbate 20 levels of 0-60% of the target level were prepared and IV admixtures were prepared in 50 mL or 100 mL IV bags from these DPs. Final polysorbate 20 levels in IV admixtures are indicated.

FIG. 4B is an alternate diagram showing the design of the study to simulate polysorbate degradation in IV admixtures. DP with polysorbate 20 levels ranging from 0 to 0.03% (w/v) were prepared, and from these DPs, IV admixtures were prepared in 50 or 100 mL IV bags. Final polysorbate 20 levels in the IV admixtures are indicated. Three IV admixtures ranging from 0.00042%-0.00048% (w/v) were prepared from the DP containing 0.03% (w/v) polysorbate 20.

FIG. 5A is a plot showing particle count by FI for mAb1 admixtures in 100 mL IV bags.

FIG. 5B is a plot showing particle count by FI for mAb1 admixtures in 50 mL IV bags.

FIG. 5C is a series of representative images of mAb1 particles found in IV admixtures with polysorbate 20 levels ≥0.0004% (w/v) in the IV admixture (top row) or ≤0.00033% (w/v) in the IV admixture (bottom row).

FIG. 6A is a plot showing the predicted relative polysorbate 20 levels in DP. 100% represents polysorbate 20 at the target concentration and 40% represents the minimum polysorbate 20 required plus a safety margin. Polysorbate 20 levels in DP and in IV admixtures predict shelf-life. The starting polysorbate 20 levels are shown as percent relative to the target concentration.

FIG. 6B shows the predicted polysorbate concentrations in IV admixtures prepared in 50 mL IV bags. The starting polysorbate 20 levels are shown as percent w/v polysorbate in the IV admixture.

FIG. 6C shows the predicted polysorbate concentrations in IV admixtures prepared in 100 mL IV bags. The starting polysorbate 20 levels are shown as percent w/v polysorbate in the IV admixture.

FIG. 6D is a plot showing a prediction of shelf-life based on polysorbate 20 levels in DP and in IV admixtures. Predicted polysorbate 20 levels in DP based on a starting level of 0.05% (w/v), calculated from the exponential decay model shown in Equation 1 are shown. The minimum polysorbate level 20 concentration required plus a safety margin is indicated by the solid line.

FIG. 6E is a plot showing a prediction of shelf-life based on polysorbate 20 levels in DP and in IV admixtures. Predicted polysorbate concentrations in IV admixtures prepared in 50 mL IV bags are shown. The line represented 0.0004% (w/v), the minimum polysorbate 20 concentration required to maintain a stable IV admixture.

FIG. 6F is a plot showing a prediction of shelf-life based on polysorbate 20 levels in DP and in IV admixtures. Predicted polysorbate concentrations in IV admixtures prepared in 100 mL IV bags are shown. The line represented 0.0004% (w/v), the minimum polysorbate 20 concentration required to maintain a stable IV admixture.

FIG. 7A is a plot showing the area for Region 1 (acidic charge variants) for mAb1 (y-axis), which was held for the amount of time indicated on the x-axis at 5° C., and analyzed by imaged capillary isoelectric focusing (iCIEF). Y-axis values indicate percentage of total area under the curve (AUC) that falls in the indicated region.

FIG. 7B is a plot showing the area for Region 2 (main charge species) for mAb1 (y-axis), which was held for the amount of time indicated on the x-axis at 5° C., and analyzed by iCIEF. Y-axis values indicate percentage of total AUC that falls in the indicated region.

FIG. 7C is a plot showing the area for Region 3 (basic charge variants) for mAb1 (y-axis), which was held for the amount of time indicated on the x-axis at 5° C., and analyzed by iCIEF. Y-axis values indicate percentage of total AUC that falls in the indicated region.

FIG. 8A is a plot showing the area for the main peak for mAb1 (y-axis), which was held for the amount of time indicated on the x-axis at 5° C., and analyzed by SE-UPLC (size-exclusion ultraperformance liquid chromatography). Y-axis values indicate the percentage of total peak area that falls in the indicated area.

FIG. 8B is a plot showing the area for the high molecular weight (HMW) area on a chromatogram for mAb1 (y-axis), which was held for the amount of time indicated on the x-axis at 5° C., and analyzed by SE-UPLC. Y-axis values indicate the percentage of total peak area that falls in the indicated area.

FIG. 8C is a plot showing the area for the low molecular weight (LMW) area on a chromatogram for mAb1 (y-axis), which was held for the amount of time indicated on the x-axis at 5° C., and analyzed by SE-UPLC. Y-axis values indicate the percentage of total peak area that falls in the indicated area.

FIG. 9A is a plot showing the area for Region 1 (acidic charge variants) for mAb1 (y-axis), which was held for the amount of time indicated on the x-axis at 5° C., and analyzed by cation exchange chromatography (CEX). Y-axis values indicate the percentage of total peak area that falls in the indicated area.

FIG. 9B is a plot showing the area for Region 2 (main charge species) for mAb1 (y-axis), which was held for the amount of time indicated on the x-axis at 5° C., and analyzed by CEX. Y-axis values indicate the percentage of total peak area that falls in the indicated area.

FIG. 9C is a plot showing the area for Region 3 (basic charge variants) for mAb1 (y-axis), which was held for the amount of time indicated on the x-axis at 5° C., and analyzed by CEX. Y-axis values indicate the percentage of total peak area that falls in the indicated area.

FIG. 10A is a plot showing the percentage of LMW area for mAb1 held for the indicated amount of time at 5° C., and analyzed by microchip capillary electrophoresis (MCE) under reduced conditions.

FIG. 10B is a plot showing the percentage of LMW area for mAb1 held for the indicated amount of time at 5° C., and analyzed by microchip capillary electrophoresis (MCE) under non-reduced conditions.

FIG. 11 is a plot showing the potency of mAb1 held for the indicated amount of time on the x-axis at 5° C., and assayed using a luciferase reporter assay.

FIG. 12A is a series of images showing particle images from FI for delivered IV admixtures containing 0.00033% (w/v) polysorbate 20.

FIG. 12B series of images showing particle images from FI for delivered IV admixtures containing 0.00025% (w/v) polysorbate 20 showing darker particles more readily detected by LO in FIG. 13A.

FIG. 13A is a plot showing particle count by FI for mAb1 admixtures prepared at t=0 (t: time).

FIG. 13B is a plot showing particle count by FI for mAb1 after overnight storage at 25° C. and 5° C. for 24 hours.

FIG. 14 is a series of plots showing a comparison of the degradation of polysorbate 20 in mAb1 research and GMP lots. All data were fit to a linear model or exponential decay model. In all cases the fit parameters were similar between the research and GMP lots. Additionally, there was little difference between the quality of the linear and exponential fits.

### DETAILED DESCRIPTION

In recent years health authorities have paid an increased level of attention to condition of use studies demonstrating the in-use stability of IV admixtures containing biologic drugs in the interest of assuring patient safety. Among the most important quality attributes to monitor for such studies of biologic drugs are potency, protein concentration and particulate analysis. Both fatty acid particles and protein particles can compromise the quality of parenteral formulations and may pose a safety risk for patients. Protein particles can pose an increased risk for immunogenicity. The presence of fatty acid particles can pose a quality risk, however, the extent of the risk to safety and efficacy is not fully understood. The main products from enzymatic degradation of polysorbate 20 are insoluble particulates composed primarily of lauric, myristic and palmitic acids. Although the presence of particles in DP is a quality concern, the risks that fatty acid particles pose to patient safety is unclear. Without wishing to be bound by theory, it is thought that the fatty acid particles found in a drug product can dissolve if diluted in saline or dextrose solutions to ≥2× dilution as their concentration falls below the solubility limit. It was also demonstrated that human serum albumin can prevent the formation of free fatty acid particles and also reverse fatty acid particles already formed. It has also been shown that fatty acid particles can be reversed using human serum.

Polysorbate 20 (also referred to as polyoxyethylene (20) sorbitan monolaurate, or Tween 20) and polysorbate 80 (also referred to as polyoxyethylene (80) sorbitan monooleate, or Tween 80) are among the most used non-ionic surfactants in biopharmaceutical protein formulations. Polysorbate 20 and polysorbate 80 improve stability by of liquid drug products (DP) comprising proteins by protecting proteins from aggregation, reducing interfacial stress, and reducing adsorption onto surfaces. However, polysorbates are known to degrade by two major pathways: auto-oxidation and enzymatic hydrolysis. Enzymatic hydrolysis of polysorbates by residual host-cell lipases during liquid DP storage leads to polysorbate degradation. Polysorbate degradation contributes to protein instability, which can result in the formation of sparingly soluble compounds such as free fatty acid (FFA) particles which negatively impact drug product quality.

Residual host-cell lipases can be present in drug products at levels below what can be detected using conventional assays, and their effects on polysorbate degradation may not be meaningful or noticeable for months or years under typical liquid storage conditions (e.g., at 2-8° C.). For example, for biologic drug products purified from Chinese Hamster Ovary cells, lipases may be present at levels that may be difficult to detect, but are still sufficient to significantly reduce the level of polysorbate in the drug formulation over the product shelf-life.

Intravenous administration of biopharmaceutical compositions such as monoclonal antibodies is one of the most commonly used routes of administration. Formulations of compositions that are administered intravenously need to be optimized not only for shelf-life stability, but also to ensure in-use stability of diluted IV admixtures. For biopharmaceutical compositions administered intravenously, the drug product, which may be stored for all or a portion of a pre-determined shelf-life, is added to a 50 mL (or larger) bag or bottle of IV infusion solution to generate an intravenous (IV) admixture. However, while the effects of polysorbate degradation on drug product quality has been studied, little is known about the resulting IV admixture quality when polysorbate degradation is observed in a drug product. Without wishing to be bound by theory, it is thought that stability of protein biopharmaceutical formulations during storage, agitation and in-use conditions can affect particular quality attributes of the proteins when formulated as IV admixtures, and during IV administration. Among the most important quality attributes to monitor for biologic drugs in IV admixtures are potency, protein concentration and particulate analysis. The ratio of antibody to polysorbate, as well has headspaces, are also thought to be important contributing factors for soluble aggregates during IV admixture storage for up to four hours. In addition, studies looking at the effect of sodium chloride and polysorbate on the aggregation propensity of a monoclonal antibody during IV administration found that more than 0.001% (w/v) polysorbate 20 was required to avoid an increase in particulate matter formation. Another study has shown that FFA particles formed in drug products by degradation of polysorbates can dissolve upon dilution into IV diluents. The interaction of proteins with components of the drug product formulation and the IV admixture formulation are complex, and the effects of IV admixture on protein potency, concentration, aggregation and the presence of particulates are poorly understood. While it is generally accepted that four hours is the maximum time a prepared IV admixture can be held before expiring without supporting microbial challenge data, it is also important to consider IV admixture storage conditions of greater than four hours to allow maximum flexibility in clinical or commercial settings.

When biopharmaceutical drug products are administered intravenously, they are typically diluted in an IV bag or bottle comprising normal saline (0.9% sodium chloride) or a 5% dextrose diluent solution. Biopharmaceutical drug formulations are frequently optimized to include stabilizers or surfactants necessary to ensure stability and quality of the drug product. Formulations for IV administration may require one or more additional stabilizers or surfactants to ensure that the protein is still stable when diluted for IV administration. The presence of host cell lipases that can degrade polysorbate, an exemplary surfactant, poses a risk not only to the stability of the drug product but also the IV admixture. Reducing the levels of polysorbates in drug product, for example through degradation during storage, can lead to an increase in free fatty acid particles in the IV admixture. While it is possible that these particles can re-solubilize upon dilution into IV diluents, or that they may not cause precipitation or increase in particle size during infusion, additional problems with the protein drug product may occur. Degradation of surfactants such as polysorbates lowers the concentration of these surfactants in the drug product, which can increase the risk of protein instability to physical stresses after dilution in IV infusion bags, such as during mixing and administration. When protein aggregates form in insufficiently stabilized IV admixtures, they pose a potential risk of immunogenic responses in the subject receiving the protein. There is therefore a need to monitor potential aggregate formation and any other changes in protein quality attributes, and attributes of drug product and IV admixture formulations that may lead to aggregate formation, such as polysorbate levels after degradation by host-cell lipases, and the ability of polysorbates to stabilize the protein against the various stresses of IV administration. The ability of the remaining polysorbate after degradation by host-cell lipases to stabilize the protein against the various stresses of IV administration should also be evaluated. In addition, the IV dosing materials such as bags, IV sets, catheters, and diluents must be evaluated for admixture compatibility across the concentration of polysorbate present in the DP, and across the lifetime of the DP shelf-life.

Demonstration of drug product compatibility with a variety of diluents and IV dosing materials is required as part of the drug development and regulatory submission process (International Conference on Harmonization guideline M4G-Common Technical Document, Quality section 3.2.P.2.6). Biopharmaceutical drug products must be compatible with the various stresses and materials encountered during IV infusion, for example, the biopharmaceutical drug products must be compatible with the effects of agitation stress, and exposure to materials present in common IV bags, IV diluents, IV sets, in-line filters and catheters. An array of analytical techniques are typically used to monitor the quality attributes of proteins in the IV admixture, such as protein concentration, purity, sub-visible particles, and potency. Of these, one important IV admixture quality attribute to monitor is sub-visible particle formation.

Common techniques used to assess insoluble particles that can arise during the IV administration process are limited. Chapter <788> of the United States Pharmacopeia (USP) Particulate Matter in Injections specifies that particles do not exceed 6000 particles ≥10 μm and 600 particles ≥25 μm per container. Particle analysis by light obscuration (LO) has generally been the preferred testing method, with microscopic particle count test by flow imaging (FI) often used as an orthogonal method for further characterization and understanding. However, translucent or irregularly shaped particles, or particles that have a similar refractive index as the matrix fluid, are not easily quantified by LO methods and therefore could result in an underestimation of the overall particle count. Orthogonal methods such as FI can be used in IV compatibility and in-use stability studies to elucidate the morphology and number of insoluble aggregates present in solution.

In cases where protein instability is observed, mitigation strategies can be taken. One strategy to improve protein stability during IV administration is to add a stabilizing solution containing additional polysorbate or other stabilizing agent (e.g. human serum albumin) to the IV admixture to maintain sufficient polysorbate levels upon dilution into the IV bag. However, this strategy may not be feasible in a commercial setting due to increased costs and complexities associated with providing a product-specific stabilizing solution. Ensuring there is sufficient polysorbate in the drug product before dilution into the IV admixture, by either minimizing polysorbate degradation by host-cell lipases, or by having polysorbate levels in the formulation at a level sufficient to maintain IV admixture quality over the drug product shelf-life, even if polysorbate degradation occurs, is preferable.

IV compatibility studies are often performed shortly after a drug product is manufactured, as well as with aged drug product. Generally, however, there is no consistent approach used when selecting aged drug product material to study compatibility and in-use stability risks. As host-cell lipases can be difficult to remove completely, a robust formulation should allow for sufficient drug product and IV admixture stability in the presence of potential host-cell lipases throughout the drug product shelf-life. Variability in residual host-cell lipases between batches of drug product may also result in varying amounts of polysorbate degradation between drug product lots. Understanding the minimum required polysorbate levels in a drug product to sufficiently stabilize a protein, such as a monoclonal antibody (mAb) during IV administration, as well as understanding how much polysorbate degradation occurs during drug product shelf-life, must be part of the drug product development program.

While new assays for polysorbate degradation are being developed, testing for polysorbate degradation as part of routine release and stability testing may remain challenging. It is thus important to understand the risk that residual host-cell lipases pose not only to the quality and stability of protein drug products, but also the risk posed to the quality and stability of the IV admixture. Degradation of polysorbate may be detected in the drug product if polysorbate analysis is on the release and stability panel. However, polysorbate analysis is currently not common for IV admixture testing. Instead, IV admixture testing is typically done in research and development laboratories, and rarely assessed in a good manufacturing practice (GMP) environment that is used to manufacture therapeutic proteins. Therefore, the appropriate amount of polysorbate to add to protein drug products to produce a stable IV admixture throughout the shelf-life of the drug product is typically a decision made during product development. Additionally, the quality of the drug product may meet all quality attributes, but the extent of polysorbate degradation may be such that impacts to quality are only observed once the biologic drug is diluted in an admixture. Furthermore, degradation of polysorbate and its impact on IV admixture stability should be considered early enough in development to allow for a proper specification setting discussion or adjustment of the formulation excipients to best suit the intended use of the product.

In general, IV admixtures should be tested with material that is close to or at the end of proposed expiration date. This is explicitly stated in European Union guidance documents, but not something explicitly outlined by the US Food and Drug Administration. The methods described herein can both inform and complement studies on aged DP by simulating aging and the effects of degraded excipients on the quality of IV admixtures. This can be used to set a product shelf-life, or specify the lower limit of polysorbate needed in the DP.

The disclosure is based on an evaluation of the effects of surfactant degradation on the quality of IV admixtures containing proteins. The inventors have simulated the effect of polysorbate degradation on protein drug product quality in an IV admixture, and used these methods to determine a minimum surfactant concentration, for example polysorbate 20, in the drug product to maintain product quality and stability in the IV admixture, as well as the minimum surfactant concentration required in the IV admixture. The methods described herein can be used to determine the minimum amount of surfactant to include in the drug product that is required to maintain stability of IV admixtures, in advance of available aged drug product at or near the end of its predicted shelf-life. The methods described herein can also be used to determine the maximum shelf-life of a drug product to be administered as an IV admixture. The methods described herein can be applied to any protein drug product intended for intravenous or parenteral administration, and can be used to minimize protein aggregation and particle formation in IV formulations. Monoclonal antibody drug products are envisaged as within the scope of the instant disclosure. The methods described herein can be used to assess the risk of particle formation in IV admixtures for biologic drugs, particularly when residual host-cell lipases may be present. The methods described herein can also be used to minimize protein aggregation and particle formation for protein formulation formulations, for example during IV studies performed during biopharmaceutical drug product development.

The methods described herein can also be used to determine the amount of surfactant, for example polysorbate 20, to include in a drug product for which product that has been aged the entire predicted shelf-life (e.g., 30 months or more) is not available. This approach can model the effect of surfactants, and predict safe levels to include in the drug product, for drug products being developed under accelerated programs that do not have batches at full predicted shelf-life.

### Definitions

As used herein “an amount of surfactant” refers to the amount of surfactant present in a solution in units of weight of surfactant per units of volume of solution. Generally, amount of surfactant is reported as percent “weight per volume (w/v)”, which refers to grams of solute in 100 milliliters of solution. As an example, percent w/v of polysorbate in a liquid antibody formulation refers to grams of polysorbate divided by the milliliters of solution, multiplied by 100 to get the % w/v. However, the person of ordinary skill in the art will understand that, given the volume of solution and weight of surfactant involved, other units (e.g., liters, or milligrams), may be appropriate.

A “target amount,” or “initial amount” of surfactant refers to the amount of surfactant included in the liquid pharmaceutical composition as originally formulated, i.e. when produced as a formulated drug substance. Frequently, formulated drug substances must follow good manufacturing practices (GMP) which specify a set amount of surfactant to include in in the formulated drug substance. The target amount, or initial amount of surfactant can thus be thought of in absolute terms, i.e. as the specified % w/v of surfactant in the composition. Alternatively, or in addition, the target or initial amount of surfactant can be described in terms that relate it to the amount of surfactant specified for the liquid pharmaceutical composition. For example, if the specifications of the liquid pharmaceutical composition calls for 100 units of surfactant, but only 95 units are added, the initial amount of surfactant can be described as 95%.

“Excipient” or “diluent” refers to an inactive substance that serves as, or is present in, the vehicle or medium for an active substance such as a small molecule or biologic. Excipients are included in formulations such as drug substance or dug product formulations. Excipients can serve many purposes, including long-term stabilization, adding bulk to the formulation (e.g., fillers or diluents), altering viscosity or solubility, or conferring therapeutic enhancements on the active substance. Depending on the active substance and the formulation, excipients can be liquid or solid. Exemplary excipients or diluents of the disclosure include excipients or diluents suitable for generating IV admixtures from a drug substance.

“Surfactants” are a class of excipients that decrease the surface tension between two liquids, a liquid and a solid, or gas and a liquid. Exemplary surfactants include detergents which, when added to a liquid, reduce its surface tension, and increase its spreading and wetting properties. For compositions comprising proteins, surfactants can act to stabilize the protein in the composition by protecting proteins from aggregation, reducing interfacial stress, and reducing adsorption onto surfaces. Surfactants can be non-ionic, amphoteric, cationic, or anionic. In general, anionic surfactants are organic salts which dissociate at high pH to form a long-chain anion with surface activity. Exemplary anionic surfactants contain carboxylate, sulfonate, or sulfate groups, e.g. e.g. sodium lauryl sulfate and docusate sodium. Cationic surfactants are positively charged substances, and may be used as preservatives. Exemplary cationic surfactants include phosphatides, e.g., phosphatidylcholine. Amphoteric surfactants (sometimes called zwitterionic) exhibit both anionic and cationic dissociations in a single surfactant molecule. Exemplary amphoteric surfactants include betaines (e.g. sulfobetaine) and natural substances such as amino acids and phospholipids. Non-ionic surfactants contain hydrophilic groups that do not ionize at any pH value. Exemplary non-ionic surfactants include esters such as polyoxyethylene sorbitan fatty acid esters (polysorbates) and the like. Further examples of non-ionic surfactants include polyglycerol alkyl ethers, glucosyl dialkyl ethers, crownethers, ester-linked surfactants, polyoxyethylene alkyl ethers, Brij, Spans (sorbitan esters) and Tweens (Polysorbates). Exemplary polysorbates include polysorbate 80 (also known as Tween 80, or polyoxyethylene (80) sorbitan monooleate), and polysorbate 20 (also known as Tween 20, or polyoxyethylene (20) sorbitan monolaurate).

“Drug substance” or “DS”, as used herein, refers to the main (or active) ingredient or ingredients in a drug product, defined below. Drug substances are also referred to as pharmaceutical ingredients, active pharmaceutical ingredients, and pharmacologic substances. An active ingredient is any component of a drug product intended to furnish pharmacological activity or other direct effect in the diagnosis, cure, mitigation, treatment, or prevention of disease, or to affect the structure or any function of the body of humans or other animals. Drug substances include small molecules and biologics.

“Formulated drug substance” or “FDS” refers to a drug substance that has been formulated into a composition that is ready for storage, shipment, or processing into its final labeled and packaged form. Formulated drug substances are frequently generated by combining a drug substance with one or more pharmaceutically acceptable diluents or excipients, including, but not limited to, suitable surfactants, fillers, extenders, diluents, wetting agents, solvents, emulsifiers, preservatives, flavors, absorption enhancers, sustained-release matrices, and coloring agents. Exemplary formulated drug substances include the liquid pharmaceutical compositions described herein.

“Drug product” or “DP”, as used herein, refers to a finished dosage form, e.g., tablet, capsule, or solution, that contains a drug substance, generally, but not necessarily, in association with one or more other ingredients.

“Shelf-life”, as used herein, refers to the length of time that a composition as described herein can be stored before becoming unfit for use. For example, the shelf-life may be the maximum amount of time that a liquid pharmaceutical composition can be stored at 2-8° C. before an IV admixture of the liquid pharmaceutical composition has more than 6000 particles ≥10 μm and 600 particles ≥25 μm per container. An exemplary shelf-life of a liquid pharmaceutical composition comprising a protein, as described herein, is about 36 months. However, the person of ordinary skill in the art will appreciate that shelf-life depends on the protein contained in the liquid pharmaceutical composition, the non-active ingredients, the container, storage conditions and the life.

“End of shelf-life” of a composition refers to a composition that has been stored for its entire shelf-life, or for nearly its entire shelf-life. As an example, a composition can be thought to be at the end of its shelf-life, if the shelf-life is 36 months, and the composition has been stored, since its formulation (or the start of its shelf-life) for a time that is within 1 week, 2 weeks, 3 weeks or 4 weeks of the shelf-life. As an additional example, a composition can be thought to be at the end of its shelf-life if it has been stored for at least 90%, at least 95%, at least 96%, at least 97%, at least 98% at least 99%, or 100% of its shelf-life.

“Storage conditions” refer to conditions under which liquid pharmaceutical compositions and IV admixtures thereof are typically stored, in accordance with manufacturer's guidelines and standard industry practice. Liquid pharmaceutical compositions comprising therapeutic proteins are typically stored at 2-8° C., and optionally protected from light or with additional guidance regarding container type. Storage of liquid pharmaceutical compositions under these conditions can range from 3 to 60 months. For example, liquid pharmaceutical compositions can be stored at 2-8° C. for about 3, 6, 9, 12, 18, 24, 30, 36, 42, 48, 54 or 60 months. Following dilution of the liquid pharmaceutical composition into a container such as an IV bag to generate an IV admixture, the IV admixture may be held for a day (e.g., 12 to 28 hours, or about 18 to 24 hours) at 2-8° C., followed by room temperature storage for 6 to 10 hours, for example when being transported or handled in clinical settings prior to administration to a subject.

As used herein, the term “subject” includes any human or nonhuman animal. The term “nonhuman animal” includes all vertebrates, for example, but not limited to, mammals and non-mammals, such as nonhuman primates, sheep, dogs, cats, horses, cows, chickens, amphibians, reptiles, etc.

The terms “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence. The term also includes protein-drug conjugates.

The phrase “pharmaceutically acceptable” as used herein means approved by a regulatory agency of the Federal or a state government, or listed in the U.S. Pharmacopcia, European Pharmacopia or other generally recognized pharmacopeia for use in animals, and more particularly in humans.

All publications, patents, and patent applications mentioned in this specification are herein incorporated by reference to the same extent as if each individual publication, patent, or patent application was specifically and individually indicated to be incorporated by reference.

The present description sets forth numerous exemplary configurations, methods, parameters, and the like. It should be recognized, however, that such description is not intended as a limitation on the scope of the present disclosure, but is instead provided as a description of exemplary embodiments. Embodiments of the present subject matter described above may be beneficial alone or in combination, with one or more other aspects or embodiments. Without limiting the foregoing description, certain non-limiting embodiments of the disclosure are provided below. As will be apparent to those of skill in the art upon reading this disclosure, each of the individually numbered embodiments may be used or combined with any of the preceding or following individually numbered embodiments. This is intended to provide support for all such combinations of embodiments and is not limited to combinations of embodiments explicitly provided below.

### Methods

The disclosure provides methods of determining a target (or initial) amount of surfactant to include in a liquid pharmaceutical composition comprising a protein and a surfactant, which is suitable for administration as an intravenous (IV) admixture. Alternatively, the methods described herein can be used to determine the shelf-life of the liquid pharmaceutical composition. As surfactants can degrade over time, by determining the degradation rate of the surfactant, the shelf-life of the liquid pharmaceutical composition, and the minimum amount of surfactant needed to maintain stability of the protein when the liquid pharmaceutical composition is formulated as an IV admixture, it is possible to determine the necessary amount of surfactant (the target amount) to include in the liquid pharmaceutical composition at the time of formulation so that sufficient surfactant remains at the end of shelf-life. For example, compositions are generally considered safe for administration to human subjects via IV administration when there are less than 6000 particles greater than 10 μm and less than 600 particles greater than 25 μm present per container. Thus, the target amount of surfactant to include at time of formulation comprises an amount of surfactant such that when the liquid pharmaceutical composition is formulated as an IV admixture at the end of its shelf-life, there are less than 6000 particles greater than 10 μm and less than 600 particles greater than 25 μm present per container of IV admixture. Similarly, it is possible, from the minimum amount of surfactant required to maintain stability, the degradation rate of the surfactant, and the initial amount of surfactant included at formulation, to determine shelf-life of the liquid pharmaceutical composition.

Accordingly, the disclosure provides methods of determining a target amount of surfactant in a liquid pharmaceutical composition comprising a protein whereby stability of the protein is maintained in an IV admixture comprising the liquid pharmaceutical composition. In some embodiments, the methods comprise (a) measuring a rate of degradation the surfactant in the liquid pharmaceutical composition; (b) determining a minimum amount of surfactant in the liquid pharmaceutical composition whereby stability of the protein is maintained in an IV admixture comprising the liquid pharmaceutical composition, and (c) based on the rate of degradation from step (a), a shelf-life of the liquid pharmaceutical composition, and the minimum amount of surfactant from step (b), determining the target amount of surfactant in the liquid pharmaceutical composition whereby stability of the protein is maintained in the IV admixture when the IV admixture is formulated at the end of the shelf-life of the liquid pharmaceutical composition.

In some embodiments, the methods comprise: (i) generating a plurality of liquid pharmaceutical compositions, wherein liquid pharmaceutical compositions in the plurality differ by an amount of the surfactant present in the liquid pharmaceutical compositions; (ii) generating a plurality of IV admixtures from the plurality of liquid pharmaceutical compositions by mixing each liquid pharmaceutical composition with a diluent suitable for intravenous (IV) administration in a container; (iii) simulating intravenous delivery of the plurality of IV admixtures to a subject; (iv) measuring particles per container of IV admixture for IV admixtures in the plurality; (v) determining a minimum amount of surfactant whereby an amount of particles per container of IV admixture does not exceed a mandated amount; and (vi) based on a shelf-life of the liquid pharmaceutical composition, the minimum amount of surfactant from step (v), and a degradation rate of the surfactant, determining a target amount of surfactant in the liquid pharmaceutical composition whereby stability of the protein is maintained in the IV admixture when the IV admixture is formulated at the end of the shelf-life of the liquid pharmaceutical composition.

The disclosure provides methods of determining a maximum amount of time a liquid pharmaceutical composition comprising a protein and a surfactant can be stored (shelf-life). In some embodiments, the methods comprise (a) measuring a rate of degradation of the surfactant in the liquid pharmaceutical composition; (b) determining a minimum amount of surfactant in the liquid pharmaceutical composition whereby stability of the protein is maintained in an IV admixture comprising the liquid pharmaceutical composition; and (c) based on the rate from degradation of step (a), an initial amount of surfactant in the liquid pharmaceutical composition, and the minimum amount of surfactant from step (b), determining the maximum shelf-life of the liquid pharmaceutical composition whereby stability of the protein is maintained in the IV admixture when the IV admixture is formulated at the end of the shelf-life of the liquid pharmaceutical composition.

In some embodiments, the methods comprise (i) generating a plurality of liquid pharmaceutical compositions, wherein liquid pharmaceutical compositions in the plurality differ by an amount of the surfactant present in the liquid pharmaceutical compositions; (ii) generating a plurality of IV admixtures from the plurality of liquid pharmaceutical compositions by mixing each liquid pharmaceutical composition with a diluent suitable for intravenous (IV) administration in a container; (iii) simulating intravenous delivery of the plurality of IV admixtures to a subject; (iv) measuring particles per container of IV admixture for IV admixtures in the plurality; (v) determining a minimum amount of surfactant whereby an amount of particles per container of IV admixture does not exceed a mandated amount; and (vi) based on a rate of degradation of the surfactant, the minimum amount of surfactant from step (v), and an initial amount of surfactant in the liquid pharmaceutical composition, determining the maximum shelf-life of the liquid pharmaceutical composition whereby stability of the protein is maintained in the IV admixture when the IV admixture is formulated at the end of the shelf-life of the liquid pharmaceutical composition.

In some embodiments, the mandated amount of particles comprises no more than 6000 particles greater than 10 μm and no more 600 particles greater than 25 μm per container. However, the person of ordinary skill in the art will appreciate that, depending on precise delivery mechanism, the container, and other variables, other particle amounts that are more or less stringent with respect to size and number may be acceptable. For example, the mandated amount may comprise no than 3,000 particles, 4,000 particles, 5,000 particles, 6,000 particles, 7,000 particles, 8,000 particles, 9000 particles or 10,000 particles greater than 10 μm per container. As a further example, the mandated amount may comprise no more than 300, 400, 500, 600, 700, 800, 900 or 1000 particles greater than 25 μm per container.

Measuring a rate of degradation for the surfactant in the liquid pharmaceutical composition can be done by any suitable methods known in the art. In some embodiments, measuring the rate of degradation of the surfactant comprises (i) determining an initial amount of surfactant in the amount in the pharmaceutical composition; (ii) holding the pharmaceutical composition for at least a first amount of time; (iii) determining at least a second amount of surfactant in the pharmaceutical composition; and (iv) applying a model of surfactant concentration over time. In some embodiments, measuring a rate of degradation for the surfactant in the liquid pharmaceutical composition comprising repeating steps (ii) and (iii) at least once. In some embodiments, the methods comprise repeating steps (ii) and (iii) at least once. In some embodiments, the methods comprise repeating steps (ii) and (iii) at least 1×, at least 2×, at least 3×, at least 4×, at least 5×, at least 6×, at least 7×, at least 8×, at least 9× or at least 10×. In some embodiments, the amount of time comprises at least 2 weeks, at least 1 month, at least 2 months, at least 3 months, at least 4 months, at least 5 months, at least 6 months, or at least 8 months. For example, the liquid pharmaceutical composition may be held for 2, 3, 4, 5, 6, 8, 10, 12, 18, 24, 30, or 36 months, and sampled monthly (i.e., the at least a first amount of time is one month). As a further example, the liquid pharmaceutical composition may be held for 2, 3, 4, 5, 6, 8, 10, 12, 18, 24, 30, or 36 months and sampled every other month (i.e., the at least a first amount of time is 2 months).

The amount of surfactant present initially in the pharmaceutical composition can be determined empirically, or calculated by adding a known amount of surfactant to the pharmaceutical composition. If determined empirically, the amount of surfactant present in the composition can be measured by any suitable methods known in the art. For example, high-performance liquid chromatography (HPLC), coupled with charged aerosol detection, can be used to detect non-volatile and semi-volatile compounds, and can be used to detect surfactants such as polysorbates. Following formulation, the pharmaceutical composition is held for at least a first period time, followed by measuring the amount of surfactant. The conditions under which the pharmaceutical composition is held should model generally accepted storage conditions, for example 2-8° C., and protected from light, and the pharmaceutical composition should be held for a sufficient length of time that degradation of the surfactant is detectable. For example, for antibody formulations, degradation of surfactant can be detected at least at 2 months, 3 months, 4 months, 5 months, 6 months, 7 months, 8 months, 9 months, 10 months, 11 months, 12 months, 18 months, 24 months, 30 months, 36 months, 42 months, 48 months, 54 months or 60 months. The person of ordinary skill in the art will appreciate that repeated sampling, for example every month, every 2 months, every 3 months, and the like, can reduce error and more accurately resolve the degradation rate of the surfactant in the liquid pharmaceutical composition.

In some embodiments, the methods comprise applying a model of surfactant concentration over time. In some embodiments, the degradation rate of the surfactant is linear, and the model is a linear model. In some embodiments, the model comprises an equation of

$$y = mx + b ,$$

wherein y is the amount of surfactant in the formulation at time point x, x is the time (in months) since initial formulation of the pharmaceutical composition, and b is the amount of surfactant in the initial pharmaceutical composition.

In some embodiments, the model comprises an exponential decay model. Exponential decay formulas known in the art, any of which may be suitable for modeling surfactant degradation in the liquid pharmaceutical composition depending on the particular liquid pharmaceutical composition and the manner in which the surfactant degrades. Selection of an appropriate formula to fit to the data will be within the skill of the person of ordinary skill in the art.

As an example, an exponential decay formula can take the form of:

$$f ( t ) = {ab}^{x}$$

where f(x) is the amount surfactant in the composition at time x (typically in months), a is the initial amount of surfactant, and b is a decay factor.

As a further example, an exponential decay formula can take the form of:

$$f ( x ) = {a ( 1 - r )}^{x} ,$$

where (x) is the amount surfactant in the composition at time x (typically in months), a is the initial amount of surfactant, and r is the rate of decay.

As a still further example, an exponential decay formula can take the form of:

$$P = {P}_{0} {e}^{- kx} ,$$

where P₀ is the initial amount of surfactant, P is the amount of surfactant at time x (typically in months), e is the exponential constant (Exp, or Exponential, also called Euler's number), and k is a proportionality constant.

As a still further example, an exponential decay formula can take the form of:

$$y = a x {e}^{bx} ,$$

where y is the amount of surfactant in the liquid pharmaceutical composition at time x, a is the scale factor (a constant), and b is the growth rate.

In some embodiments, the methods comprise applying a model of surfactant concentration over time. In some embodiments, the model is an exponential decay model. In some embodiments, the model comprises an equation of:

$$Surfactant \% ( w / v ) = a * Exp ( b * time , months )$$

where a is the scale, b is the growth rate, and “Exp” stands for “Exponential” or “e.”

In some embodiments, generating the model comprises regression analysis. Regression analysis is a set of statistical processes for estimating the relationships between a dependent variable (often called the ‘response’ variable) and an more independent variables. One common form of regression analysis is linear regression, in which the person of ordinary skill in the art finds the line that most closely fits the data according to a specific mathematical criterion. For example, the method of ordinary least squares computes the unique line that minimizes the sum of squared differences between the true data and that line).

In some embodiments, fitting the model comprises linear regression. Linear regression is a linear approach for modelling the relationship between a scalar response variable and one or more explanatory variables. The case of one explanatory variable is called simple linear regression. In linear regression, the relationships are modeled using linear predictor functions whose unknown model parameters are estimated from the data. Such models are called linear models.

Linear regression was the first type of regression analysis to be studied rigorously, and to be used extensively in practical applications. This is because models which depend linearly on their unknown parameters are easier to fit than models which are non-linearly related to their parameters and because the statistical properties of the resulting estimators are easier to determine.

In some embodiments, the regression analysis comprises polynomial regression. Polynomial regression is a form of regression analysis in which the relationship between the independent variable and the dependent variable is modelled as an nth degree polynomial. Polynomial regression fits a nonlinear relationship between the value of independent variable and the corresponding conditional mean of the dependent variable. Although polynomial regression fits a nonlinear model to the data, as a statistical estimation problem it is linear, in the sense that the regression function is linear in the unknown parameters that are estimated from the data. For this reason, polynomial regression is considered to be a type of multiple linear regression.

Polynomial regression models can be fit using the method of least squares. The least-squares method minimizes the variance of the unbiased estimators of the coefficients, under the conditions of the Gauss-Markov theorem.

In some embodiments, fitting the model comprises curve fitting. Curve fitting is the process of constructing a curve, or mathematical function, that has the best fit to a series of data points. Curve fitting can involve either interpolation, where an exact fit to the data is required, or smoothing, in which a “smooth” function is constructed that approximately fits the data. Curves can be extrapolated, i.e. extended beyond the range of the observed data, although extrapolated curves are subject to a degree of uncertainty.

In some embodiments, fitting the model comprises fitting an exponential decay model. In some embodiments, fitting the exponential decay model comprises using nonlinear regression.

Fitting the model can be carried out using any suitable program known in the art, for example Microsoft excel, MATLAB, R or JMP 16 (JMP Statistical Discovery, LLC).

In some embodiments, determining a minimum amount of surfactant in the liquid pharmaceutical composition whereby stability of the protein is maintained in an IV admixture comprises (i) generating a plurality of liquid pharmaceutical compositions comprising equivalent amounts of the protein, wherein liquid pharmaceutical compositions in the plurality differ by an amount of the surfactant present in the liquid pharmaceutical compositions; (ii) generating a plurality of IV admixtures from the plurality of liquid pharmaceutical compositions by mixing each liquid pharmaceutical composition with a diluent suitable for intravenous (IV) administration in a container; (iii) simulating intravenous delivery of the plurality of IV admixtures to a subject; (iv) collecting samples of the plurality of IV admixtures; (v) measuring particles per container of IV admixture for IV admixtures in the plurality; and (vi) determining the minimum amount of surfactant whereby an amount of particles per container of IV admixture does not exceed a mandated amount per container.

In some embodiments, compositions in the plurality of liquid pharmaceutical compositions differ substantially only by the amount of surfactant. Compositions in the plurality comprise the same, or minimal variation in, the concentrations of the protein, and inactive ingredients. The plurality of liquid pharmaceutical compositions are used to generate a plurality of IV admixtures, where the same, or substantially the same, amount of each pharmaceutical composition is mixed with the same, or substantially the same, amount of IV infusion solution at the same ratio, in the same type of container (i.e., the same make or model, from the same manufacturer), to minimize variables other than surfactant concentration. The resulting plurality of IV admixtures thus differ with respect surfactant concentration, and model the effects of different amounts of surfactant degradation that can be seen over the shelf-life of the liquid pharmaceutical composition. The IV admixtures are then held and pumped into receptacles under conditions that simulate formulating the IV admixture and administering the IV admixture to a subject in a clinical setting. In some embodiments, simulating intravenous delivery of the plurality of IV admixtures to a subject comprises: (i) incubating the plurality IV admixtures for a first period of time at 2-8° C.; (ii) incubating for a second period of time at 21-26° C.; and (iii) pumping the plurality IV admixtures into receptacles. In some embodiments, the first period of time comprises 20 to 28 hours, 22 to 26 hours, 22 to 24 hours, or 18 to 24 hours. In specific embodiments, the first period of time comprises 24 hours. In some embodiments, the second period of time comprises 4 to 12 hours, or 6-10 hours. In specific embodiments, the second period of time comprises 8 hours. In some embodiments, the second period of time comprises at least 1 hour, at least 2 hours, at least 3 hours, at least 4 hours, at least 5 hours, at least 6 hours, at least 7 hours, at least 8 hours, at least 9 hours, at least 10 hours, at least 11 hours, or at least 12 hours. In some embodiments, incubation during the second period of time takes place at 25° C.

In some embodiments, pumping the plurality of IV admixtures into receptacles comprises (i) attaching the plurality of containers to catheters and filters; (ii) holding the plurality of attached containers at about 21-26° C. for about 60 minutes; (iii) connecting the plurality of containers to IV pumps; and (iv) pumping the plurality of IV admixtures through the catheters and filters into receptacles at a rate of between 25 and 100 mL/hour. Suitable IV infusion sets, comprising catheters, filters, and IV pumps are described in more detail below. In some embodiments, the plurality of containers attached to catheters and filters are held at 25° ° C.

In some embodiments, the IV admixtures attached to catheters and filters are primed using gravity, and then a pump is attached, and the IV admixture is pumped through an IV infusion set (or at least a partial IV infusion set, simulating mechanical stress during administration) into a receptacle at a rate of between 25 and 100 mL/hour. In some embodiments, the IV admixture is pumped at a rate of between 10 and 150 mL per hour. In some embodiments, the IV admixture is pumped at a rate of between 25 and 100 mL per hour. In some embodiments, the IV admixture is pumped at a rate of between 25 and 75 mL per hour. In some embodiments, for example when the IV admixture is in a 50 mL IV bag, the IV admixture is pumped at a rate of 50 mL per hour. In some embodiments, for example when the IV admixture is in a 100 mL IV bag, the IV admixture is pumped at a rate of 100 mL per hour. In alternative embodiments, the IV admixture is allowed to flow through the IV infusion set using a gravity-based system, for example at a rate of 5, 10, 15, 20, 25 or 30 drops per minute.

In some embodiments, determining the target amount of surfactant to include in the liquid pharmaceutical composition comprises calculating a target amount based on the rate of degradation of the surfactant, the shelf-life of the liquid pharmaceutical composition, and the minimum amount of surfactant suitable for IV admixture as determined using the methods described herein. For example, if the amount of surfactant present in the liquid pharmaceutical composition is determined by the linear model y=mx+b, where y is the amount of surfactant in the formulation at time point x (in months) since initial formulation of the pharmaceutical composition, then b is the target amount of surfactant to include at time=0. If the shelf-life is, for example, x months, and the rate of degradation (m) and minimum acceptable amount of surfactant at time x months are determined experimentally as described herein, the skilled artisan can readily calculate the initial amount of surfactant (b) by applying the equation: b=y−mx, with the appropriate values for y, m and x. Similarly, the skilled artisan can readily calculate shelf-life (x) from the initial amount of surfactant (b), and the rate of change (m).

In some embodiments, the surfactant comprises polysorbate 20, and the degradation rate is between 0.0001% (w/v) and 0.0005% (w/v) per month. In some embodiments, the degradation rate comprises 0.00031% (w/v) per month.

In some embodiments, the surfactant comprises polysorbate 20, and the liquid pharmaceutical composition comprises an amount of surfactant sufficient to produce an IV admixture comprising 0.0004% (w/v) or more polysorbate 20 when the liquid pharmaceutical composition is mixed with a suitable diluent at the end of its shelf-life to produce the IV admixture. For example, if the liquid pharmaceutical composition is diluted into the IV solution at a ratio of 1:100, for the IV admixture to contain 0.0004% (w/v) polysorbate, the liquid pharmaceutical composition must contain 0.04% (w/v) polysorbate. Based on the degradation rate and shelf-life of the liquid pharmaceutical composition, a minimum starting amount of polysorbate in the liquid pharmaceutical composition to produce a liquid pharmaceutical composition with 0.04% (w/v) or 0.02% (w/v) polysorbate at the end of its shelf-life can be determined using the methods described herein.

In some embodiments, the shelf-life of the liquid pharmaceutical composition is between 6 and 60 months, inclusive of the endpoints. In some embodiments, the shelf-life of the liquid pharmaceutical composition is between 12 and 54 months, inclusive of the endpoints. In some embodiments, the shelf-life of the liquid pharmaceutical composition is between 18 and 48 months, inclusive of the endpoints. In some embodiments, the shelf-life of the liquid pharmaceutical composition is between 24 and 40 months, inclusive of the endpoints. In some embodiments, the shelf-life of the liquid pharmaceutical composition is between 30 and 36 months, inclusive of the endpoints. In some embodiments, the shelf-life of the liquid pharmaceutical composition is about 6 months, 12 months, 18 months, 30 months, 36 months, 42 months, 48 months, 54 months or 60 months. In some embodiments, the shelf-life of the liquid pharmaceutical composition is 24 months. In some embodiments, the shelf-life of the liquid pharmaceutical composition is 30 months. In some embodiments, the shelf-life of the liquid pharmaceutical composition is 36 months.

In some embodiments, the shelf-life of the liquid pharmaceutical composition is determined empirically using the methods described herein. In some embodiments, the shelf-life comprises the maximum shelf-life wherein, when the liquid pharmaceutical composition is diluted into suitable diluent to form the IV admixture, the amount of particles per container of IV admixture does not exceed a mandated amount as described herein, for example more than 6000 particles greater than 10 μm and 600 particles greater than 25 μm per container.

### IV Admixtures

The disclosure provides methods of determining an amount surfactant in a liquid pharmaceutical composition suitable for use in an intravenous (IV) admixture, or determining the shelf-life of said liquid pharmaceutical composition.

As used herein, an “IV admixture” refers to a composition suitable for IV administration to a patient without further formulation. IV admixtures can be created by mixing the liquid pharmaceutical compositions described herein with a suitable diluent or excipient. For example, liquid pharmaceutical compositions of the disclosure can be added to a 50 mL or larger IV bag or bottle of IV fluid. Due to the overfill associated with IV bags, a volume of diluent equal to the volume of liquid pharmaceutical composition to be added to the IV bag can be removed prior to the addition of the liquid pharmaceutical composition.

Suitable IV infusion solutions for use as diluents or excipients will be known to persons of ordinary skill in the art. An exemplary IV infusion solution comprises normal saline, e.g. 0.9% sodium chloride. A further exemplary IV infusion solution comprises dextrose, e.g. 5% dextrose. Suitable IV infusion solutions are commercially available, optionally pre-packaged into containers suitable for IV administration. For example, USP sterile, nonpyrogenic and isotonic 0.9% sodium chloride solutions prepackaged in 50, 100, 150, 250, 500 and 1000 mL IV bags are available from Braun. 0.9% sodium chloride solutions and 5% dextrose solutions, packaged in IV suitable containers, are also available from Baxter Hospital Products and Hospira. Thus, depending on the volume of IV admixture to be administered the subject, the protein in the liquid pharmaceutical composition, and the particulars of the IV administration set, the skilled artisan will be able to select a suitable IV infusion solution and container in which to prepare the IV admixture.

In some embodiments, the container comprising the IV admixture comprises an IV bag. In some embodiments, the IV bag comprises a 50 mL, 100 mL, or 150 mL IV bag. In some embodiments, the IV bag comprises a 50 mL IV bag. In some embodiments, the IV bag comprises a 100 mL IV bag. In some embodiments, the IV bag comprises a 150 mL IV bag. In some embodiments, the IV bag comprises a polyvinyl chloride (PVC) IV bag, a polyolefin (PO) IV bag or an ethyl vinyl alcohol (EVA) IV bag. In some embodiments, the IV bag is pre-filled with a suitable volume of IV infusion solution as described supra. In some embodiments, the IV bag comprises a polyvinyl chloride (PVC) IV bag pre-filled with a suitable volume of IV infusion solution as described supra.

In some embodiments, the container comprising the IV admixture comprises an IV bottle.

Following preparation of the IV admixture in a suitable container, the container is connected to an IV infusion set for administration to a subject. In some embodiments, the IV infusion set comprises a pump, such as a large volumetric pump, that controls the rate of administration of the IV admixture. In some embodiments, the IV admixture is administered to the subject at a rate of between 10 and 150 mL per hour. In some embodiments, the IV admixture is administered to the subject at a rate of between 25 and 100 mL per hour. In some embodiments, the IV admixture is administered to the subject at a rate of between 25 and 75 mL per hour. In some embodiments, for example when the IV admixture is in a 50 mL IV bag, the IV admixture is pumped at a rate of 50 mL per hour. In some embodiments, for example when the IV admixture is in a 100 mL IV bag, the IV admixture is pumped at a rate of 100 mL per hour. In alternative embodiments, the IV admixture is administered the subject using a gravity-based system, for example at a rate of 5, 10, 15, 20, 25 or 30 drops per minute.

Suitable IV infusion sets are known to persons of ordinary skill in the art. An exemplary IV infusion set connects the container comprising the IV admixture via a sterile spike to a drip chamber. The drip chamber may be connected to a back check valve and sterile tubing. A roller clamp attached to the tubing can be used to help control the rate of infusion, or stop administration of the IV admixture. The tubing connects to a catheter inserted into a vein of the subject. In some cases, the IV infusion set comprises an in line filter, for example a 0.2 micron or 1.2 micron filter, that removes particulates, other impurities and/or air bubbles from the IV admixture. The filter can be located, for example, in the drip chamber, before the drip chamber, or after the drip chamber. A further exemplary IV infusion set comprises a Luer-lock inlet connecting the container comprising the IV admixture to tubing, and the tubing is connected to the inline filter, a flow regulation device, a Luer-lock adaptor and a catheter inserted into a vein of the subject. IV sets can be purchased from commercial sources. For example, suitable PVC with di-(2-ethylhexyl) phthalate (DEHP) IV infusion sets are available from Alaris/Becton, Dickinson and Company/CareFusion and Baxter International. 20 or 22 G catheters and 0.2 μm polyethersulfone (PES) filters suitable for the methods described herein are available from Alaris/Becton, Dickinson and BD/CareFusion.

### Liquid Pharmaceutical Compositions

The disclosure provides liquid pharmaceutical compositions comprising a protein and a surfactant, and methods of determining the amount of surfactant contained therein, or the shelf-life of the liquid pharmaceutical compositions. Any suitable liquid pharmaceutical composition is envisaged as within the scope of the instant disclosure.

Any surfactants suitable for use with therapeutic proteins in liquid pharmaceutical compositions are envisaged as within the scope of the instant disclosure.

Exemplary surfactants are described in US20220281988, the contents of which are incorporated by reference herein in their entirety.

In some embodiments, the surfactant comprises a non-ionic, amphoteric, cationic, or anionic surfactant. In some embodiments, the non-ionic surfactant comprises a sorbitan fatty acid ester, a glycerin fatty acid ester, a polyglycerol fatty acid ester, a polyoxyethylene sorbitan fatty acid ester (polysorbate), a polyoxyethylene sorbitol fatty acid ester, a polyoxyethylene glycerin fatty acid ester, a polyethylene glycol fatty acid ester, a polyoxyethylene alkyl ether, a polyoxyethylene polyoxypropylene alkyl ether, a 1,4-polyoxyethylene alkyl phenyl ether, polyoxyethylene hardened castor oil, a polyoxyethylene bees wax derivative, a polyoxyethylene lanolin derivative, or a combination thereof.

In some embodiments, the surfactant comprises a polysorbate, for example polysorbate 20, 40, 60, 65, 80, 81 or 85, or a combination thereof. In some embodiments, the polysorbate comprises polysorbate 20. In some embodiments, the surfactant comprises polysorbate 80. In some embodiments, the surfactant comprises a combination of polysorbate 20 and polysorbate 80.

Further examples of non-ionic surfactants include, but are not limited to, sorbitan fatty acid esters such as sorbitan monocaprylate, sorbitan monolaurate and sorbitan monopalmitate; glycerin fatty acid esters such as glycerol monocaprylate, glycerol monomyristate and glycerol monostearate; polyglycerol fatty acid esters such as decaglyceryl monostearate, decaglyceryl distearate and decarlyceryl monolinoleate; polyoxyethylene sorbitan fatty acid esters such as polyoxyethylene sorbitan monolaurate, polyoxyethylene sorbitan monooleate, polyoxyethyleuesorbitan monostearate, polyoxyethylene sorbitan monopahnitate, polyoxyethylene sorbitan trioleate and polyoxyethylene sorbitan tristearate; polyoxyethylene sorbitol fatty acid esters such as polyoxyethylene sorbitol tetrastearate and polyoxyethylene sorbitol tetra oleate; polyoxyethylene glycerin fatty acid esters such as polyoxyethylene glyceryl monostearate; polyethylene glycol fatty acid esters such as polyethylene glycol distearate; polyoxyethylene alkyl ethers such as polyoxyethylene lauryl ether; polyoxyethylene polyoxypropylene alkyl ethers such as polyoxyethylene polyoxypropylene glycol ether, polyoxyethylene polyoxypropylene propyl ether and polyoxyethylene polyoxypropylene cetyl ether; 1,4-polyoxyethylene alkyl phenyl ethers such as polyoxyethylene nonylphenyl ether; polyoxyethylene hardened castor oil such as polyoxyethylene castor and polyoxyethyene hardened castor oil (polyoxyethylene hydrogenated castor oil); polyoxyethylene bees wax derivatives such as polyoxyethylene sorbitol bees wax; polyoxyethylene lanolin derivatives such as polyoxyethylene lanolin; surfactants having an HLB of 6 to 18 such as polyoxyethylene fatty acid amides, for example, polyoxyethylene octadecanamide; anionic surfactants, for example, alkyl sulfate salts having a C₁₀-C₁₈ allyl group, such as sodium cetyl sulfate, sodium lauryl sulfate and sodium oleyl sulfate; polyoxyethylene alkyl ether sulfate salts in which the average number of moles of the added ethylene oxide units is 2 to 4 and the number of carbon atoms of the alkyl group is 10 to 18, such as polyoxyethylene sodium lauryl sulfate; alkyl sulfosuccinate salts having a C₈-C₁₈ alkyl group, such as sodium lauryl sulfosuccinate; natural surfactants such as lecithin and glycerophospholipids; sphingophospholipids such as sphingomyelin; and sucrose esters of C₁₂-C₁₈ fatty acids. These surfactants can be added to the formulation of the present invention individually, or two or more of these surfactants can be added in combination.

In some embodiments, the surfactant comprises Pluronic® (high molecular weight polyoxyalkylene ether) type surfactants, for example Pluronic® F-68 (Poloxamer 188).

The amount of non-ionic surfactant contained within the liquid pharmaceutical composition of the present invention may vary depending on the specific properties desired of the composition, as well as the particular protein contained therein. There is thus a need to determine a suitable amount of surfactant to include in the liquid pharmaceutical composition using the methods described herein.

In some embodiments, the liquid pharmaceutical composition comprises an amount of surfactant whereby, after the liquid pharmaceutical composition has been held at storage conditions for its shelf-life, an IV admixture of the liquid pharmaceutical composition comprises a level of particles that is deemed safe for administration to a subject. For example, an IV admixture of the liquid pharmaceutical composition that comprises less than 6000 particles greater than 10 μm and less than 600 particles greater than 25 μm per container of IV admixture is deemed safe for administration to human subjects.

In some embodiments, the surfactant comprises polysorbate 20, and the liquid pharmaceutical composition comprises an amount of surfactant sufficient to produce an IV admixture comprising 0.0004% (w/v) polysorbate 20 when the liquid pharmaceutical composition is mixed with a suitable diluent at the end of its shelf-life to produce the IV admixture.

In certain embodiments, the liquid pharmaceutical composition comprises 0.001%+0.0005% (w/v) to 5%+0.5% (w/v) surfactant. In certain embodiments, the liquid pharmaceutical compositions comprise 0.01%+0.005% (w/v) to 0.5%+0.25% surfactant. For example, the liquid pharmaceutical compositions may comprise about 0.001% w/v; about 0.002% w/v; about 0.003% w/v; about 0.004% w/v; about 0.005% w/v; about 0.01% w/v; about 0.02% w/v; about 0.03% w/v; about 0.04% w/v; about 0.05% w/v; about 0.06% w/v; about 0.07% w/v; about 0.08% w/v; about 0.09% w/v; about 0.1% w/v; about 0.11% w/v; about 0.12% w/v; about 0.13% w/v; about 0.14% w/v; about 0.15% w/v; about 0.16% w/v; about 0.17% w/v; about 0.18% w/v; about 0.19% v; about 0.20% w/v; about 0.21% w/v; about 0.22% w/v; about 0.23% w/v; about 0.24% w/v; about 0.25% w/v; about 0.26% w/v; about 0.27% w/v; about 0.28% w/v; about 0.29% w/v; about 0.30% w/v; about 0.35% w/v; about 0.40% w/v; about 0.45% w/v; about 0.46% w/v; about 0.47% w/v; about 0.48% w/v; about 0.49% w/v; about 0.50% w/v; about 0.55% w/v; about 0.575% w/v; about 0.60% w/v; about 0.70% w/v; about 0.80% w/v; about 0.90% w/v; about 1.0% w/v; about 1.1% w/v; about 1.2% w/v; about 1.3% w/v; about 1.4% w/v; or about 1.5% w/v surfactant. In some embodiments, the liquid pharmaceutical compositions may contain between 0.001% w/v and 10% w/v, between 0.001% w/v and 5% w/v, between 0.001% w/v and 1% w/v, between 0.001% w/v and 0.5% w/v, between 0.001% w/v and 0.1% w/v, between 0.01% w/v and 10% w/v, between 0.01% w/v and 5% w/v, between 0.01% w/v and 1% w/v, between 0.01% w/v and 0.5% w/v, between 0.01% w/v and 0.1% w/v, between 0.1% w/v and 10% w/v, between 0.1% w/v and 5% w/v, between 0.1% w/v and 1% w/v, or between 0.1% w/v and 0.5% w/v surfactant, some embodiments, the liquid pharmaceutical compositions may contain between 0.01% w/v and 0.5% w/v of surfactant. In some embodiments, the liquid pharmaceutical compositions may contain between 0.01% w/v and 0.05% w/v of surfactant. In some embodiments, the surfactant comprises polysorbate 20. In some embodiments, the surfactant comprises polysorbate 80. In some embodiments, the surfactant comprises a combination of polysorbate 20 and polysorbate 80.

Exemplary liquid pharmaceutical compositions comprising proteins are described in US 2020/0216541, the contents of which are incorporated by reference herein.

In some embodiments, a liquid pharmaceutical composition of the disclosure is an aqueous formulation. In specific embodiments, a liquid pharmaceutical composition of the disclosure is an aqueous formulation wherein the aqueous carrier is distilled water.

In some embodiments, a liquid pharmaceutical composition of the disclosure is sterile. The liquid pharmaceutical compositions of the disclosure may be sterilized by various sterilization methods, including sterile filtration, radiation and the like. In some embodiments, the liquid pharmaceutical composition is filter-sterilized with a presterilized 0.22-micron filter such as a 0.22 μm polyvinylidene fluoride (PVDF) filter. Sterile compositions for injection can be formulated according to conventional pharmaceutical practice as described in “Remington: The Science & Practice of Pharmacy”, 21st ed., Lippincott Williams & Wilkins, (2005).

In some embodiments, a liquid pharmaceutical composition of the disclosure is homogeneous.

In some embodiments, a liquid pharmaceutical composition of the disclosure is isotonic.

In some embodiments, liquid pharmaceutical compositions of the disclosure comprise common excipients and/or additives such as buffering agents, saccharides, salts and surfactants. Additionally or alternatively, the liquid pharmaceutical compositions can, in some embodiments, comprise common excipients and/or additives, such as, but not limited to, solubilizers, diluents, binders, stabilizers, salts, lipophilic solvents, amino acids, chelators, preservatives, or the like. In some embodiments, liquid pharmaceutical compositions of the disclosure can comprise other common auxiliary components, such as, but not limited to, suitable excipients, polyols, solubilizers, diluents, binders, stabilizers, lipophilic solvents, chelators, preservatives, and the like. In some embodiments, the buffering agent is selected from the group consisting of histidine, citrate, phosphate, glycine, and acetate. In some embodiments, the saccharide is selected from the group consisting of trehalose, sucrose, mannitol, maltose and raffinose. In some embodiments, the salt is selected from the group consisting of NaCl, KCl, MgCl₂, CaCl₂), or a combination thereof.

In some embodiments, liquid pharmaceutical compositions of the disclosure comprise a buffering or pH adjusting agent to control pH. In some embodiments, a liquid pharmaceutical composition of the disclosure has a pH of between about 3.0 and about 9.0, between about 4.0 and about 8.0, between about 5.0 and about 8.0, between about 5.0 and about 7.0, between about 5.0 and about 6.5, between about 5.5 and about 8.0, between about 5.5 and about 7.0, or between about 5.5 and about 6.5. In some embodiments, a liquid pharmaceutical composition of the disclosure has a pH of between 3.0 and 9.0, between 4.0 and 8.0, between 5.0 and 8.0, between 5.0 and 7.0, between 5.0 and 6.5, between 5.5 and 8.0, between 5.5 and 7.0, or between 5.5 and 6.5. In some embodiments, a formulation of the disclosure has a pH of between about 5.0 and 7.5. In some embodiments, a formulation of the disclosure has a pH of between about 5.5 and 7.0. In some embodiments, a formulation of the disclosure has a pH of between about 5.0 and 6.5. In some embodiments, a formulation of the disclosure has a pH of between about 5.5 and 6.5. In some embodiments, a liquid pharmaceutical composition has a pH of about 3.0, about 3.5, about 4.0, about 4.5, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.5, about 8.0, about 8.5, or about 9.0. In some embodiments, a liquid pharmaceutical composition of the disclosure has a pH of 3.0, 3.5, 4.0, 4.5, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.5, 8.0, 8.5, or 9.0. In specific embodiments, a liquid pharmaceutical composition of the disclosure has a pH of 5.5. In specific embodiments, a liquid pharmaceutical composition of the disclosure has a pH of 6.0. In specific embodiments, a liquid pharmaceutical composition of the disclosure has a pH of 6.5. In specific embodiments, a liquid pharmaceutical composition of the disclosure has a pH of 7.0.

In some embodiments, liquid pharmaceutical compositions of the disclosure comprise a buffering agent. Exemplary buffering agents include salts prepared from an organic or inorganic acid or base. Representative buffering agents include, but are not limited to, organic acid salts such as salts of citric acid, ascorbic acid, gluconic acid, carbonic acid, tartaric acid, succinic acid, acetic acid, or phthalic acid: Tris, tromethamine hydrochloride, or phosphate buffers. In addition, amino acid components can also function in a buffering capacity. Representative amino acid components which may be utilized in the liquid pharmaceutical compositions of the disclosure as buffering agents include, but are not limited to, glycine and histidine. In some embodiments, the buffering agent is selected from the group consisting of histidine, citrate, phosphate, glycine, and acetate. In some embodiments, the purity of the buffering agent is at least 98%, or at least 99%, or at least 99.5%. As used herein, the term “purity” in the context of histidine refers to chemical purity of histidine as understood in the art, e.g., as described in. The Merck Index, 13th ed., O'Neil et al. ed. (Merck & Co., 2001).

Buffering agents are typically used at concentrations between about 1 mM and about 200 mM or any range or value therein, depending on the desired ionic strength and the buffering capacity required. In some embodiments, the buffering agent is at a concentration of about 1 mM, or of about 5 mM, or of about 10 mM, or of about 15 mM, or of about 20 mM, or of about 25 mM, or of about 30 mM, or of about 35 mM, or of about 40 mM, or of about 45 mM, or of about 50 mM, or of about 60 mM, or of about 70 mM, or of about 80 mM, or of about 90 mM, or of about 100 mM. In some embodiments, the buffering agent is at a concentration of 1 mM, or of 5 mM, or of 10 mM, or of 15 mM, or of 20 mM, or of 25 mM, or of 30 mM, or of 35 mM, or of 40 mM, or of 45 mM, or of 50 mM, or of 60 mM, or of 70 mM, or of 80 mM, or of 90 mM, or of 100 mM. In some embodiments, the buffering agent is at a concentration of between about 5 mM and about 50 mM. In some embodiments, the buffering agent is at a concentration of between 5 mM and 20 mM.

In some embodiments, liquid pharmaceutical compositions of the disclosure comprise a carbohydrate excipient. Carbohydrate excipients can act, e.g., as viscosity enhancing agents, stabilizers, bulking agents, solubilizing agents, and/or the like. Carbohydrate excipients are generally present at between about 1% and about 99% by weight or volume. In some embodiments, the carbohydrate excipient is present at between about 0.1% and about 20%. In other embodiments, the carbohydrate excipient is present at between about 0.1% and about 15% In still further embodiments, the carbohydrate excipient is present at between about 0.1% and about 5%, or between about 1% and about 20%, or between about 5% and about 15%, or between about 8% and about 10%, or between about 10% and about 15%, or between about 15% and about 20%. In some embodiments, the carbohydrate excipient is present at between 0.1% and 20%, or between 5% and 15%, or between 8% and 10%, or between 10% and 15%, or between 15% and 20%. In some embodiments, the carbohydrate excipient is present at between about 0.1% and about 5%. In some embodiments, the carbohydrate excipient is present at between about 5% and about 10%. In some embodiments, the carbohydrate excipient is present at between about 15% and about 20%. In some embodiments, the carbohydrate excipient is present at 1%, or at 1.5%, or at 2%, or at 2.5%, or at 3%, or at 4%, or at 5%, or at 10%, or at 15%, or at 20%.

Carbohydrate excipients suitable for use in the liquid pharmaceutical compositions of the disclosure include, but are not limited, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and the like. In some embodiments, the carbohydrate excipients are selected from the group consisting of, sucrose, trehalose, lactose, mannitol, and raffinose. In some embodiments, the carbohydrate excipient is trehalose. In another specific embodiment, the carbohydrate excipient is mannitol. In other embodiments embodiment, the carbohydrate excipient is sucrose. in still further embodiments, the carbohydrate excipient is raffinose. In some embodiments, the purity of the carbohydrate excipient is at least 98%, or at least 99%, or at least 99.5%.

In some embodiments, a liquid pharmaceutical composition of the disclosure comprises a salt. In some embodiments, the salt selected is from the group consisting of: sodium chloride (NaCl), potassium chloride (KCl), calcium chloride (CaCl₂)), magnesium chloride (MgCl₂), and combinations thereof. In some embodiments, the liquid pharmaceutical composition comprises between about 10 mM and about 300 mM, between about 10 mM and about 200 mM, between about 10 mM and about 175 mM, between about 10 mM and about 150 mM, between about 25 mM and about 300 mM, between about 25 mM and about 200 mM, between about 25 mM and about 175 mM, between about 25 mM and about 150 mM, between about 50 mM and about 300 mM, between about 50 mM and about 200 mM, between about 50 mM and about 175 mM, between about 50 mM and about 150 mM, between about 75 mM and about 300 mM, between about 75 mM and about 200 mM, between about 75 mM and about 175 mM, between about 75 mM and about 150 mM, between about 100 mM and about 0.300 mM, between about 100 mM and about 200 mM, between about 100 mM and about 175 mM, or between about 100 mM and about 150 mM of a salt as described herein. In some embodiments, the liquid pharmaceutical composition comprises at least 10 mM, at least 25 mM, at least 50 mM, at least 75 mM, at least 80 mM, at least 100 mM, at least 125 mM, at least 150 mM, at least 175 mM, at least 200 mM, or at least 300 mM salt.

In some embodiments, the liquid pharmaceutical composition comprises sodium chloride (NaCl). In some embodiments, the liquid pharmaceutical composition comprises at least about 10 mM, at least about 25 mM, at least about 50 mM, at least about 75 mM, at least about 80 mM, at least about 100 mM, at least about 125 mM, at least about 150 mM, at least about 175 mM at least about 200 mM, or at least about 300 mM sodium chloride. In some embodiments, the liquid pharmaceutical composition comprises at least 10 mM, at least 25 mM, at least 50 mM, at least 75 mM, at least 80 mM, at least 100 mM, at least 125 mM, at least 150 mM, at least 175 mM at least 200 mM, or at least 300 mM sodium chloride. In some embodiments, the liquid pharmaceutical composition comprises about 10 mM, about 25 mM, about 50 mM, about 75 mM, about 80 mM, about 100 mM, about 125 mM, about 150 mM, about 175 mM, about 200 mM, or about 300 mM sodium chloride. In some embodiments, the liquid pharmaceutical composition comprises 10 mM, 25 mM, 50 mM, 75 mM, 80 mM, 100 mM, 125 mM, 150 mM, 175 mM, 200 mM, or 300 mM sodium chloride.

In some embodiments, the liquid pharmaceutical compositions of the disclosure comprise other common pharmaceutically acceptable excipients and/or additives including, but not limited to, diluents, binders, stabilizers, lipophilic solvents, preservatives, adjuvants, or the like. Commonly used excipients/additives, such as pharmaceutically acceptable chelators (for example, but not limited to, EDTA, DTPA or EGTA) can optionally be added to the formulations of the disclosure to reduce aggregation. These additives are particularly useful if a pump or plastic container is used to administer the formulation in an IV admixture.

Preservatives, such as phenol, m-cresol, p-cresol, o-cresol, chlorocresol, benzyl alcohol, phenyl mercuric nitrile, phenoxy ethanol, formaldehyde, chlorobulanol, magnesium chloride (for example, but not limited to, hexahydrate), alkylparaben (methyl, cthyl, propyl, butyl and the like), benzalkonium chloride, benzethonium chloride, sodium dehydroacetate and thimerosal, or mixtures thereof can optionally be added to the liquid pharmaceutical compositions of the disclosure at any suitable concentration such as between about 0.001% to about 5%, or any range or value therein. The concentration of preservative used in the liquid pharmaceutical compositions of the disclosure is a concentration sufficient to yield an anti-microbial effect. Such concentrations are dependent on the preservative selected and are readily determined by the person of ordinary skill in the art.

Other contemplated excipients/additives, which may be utilized in the liquid pharmaceutical compositions of the disclosure include, for example, antimicrobial agents, antioxidants, antistatic agents, lipids such as phospholipids or fatty acids, steroids such as cholesterol, protein excipients such as serum albumin (human serum albumin (HSA), recombinant human albumin (rHA), gelatin, casein, salt-forming counterions such as sodium and the like. These and additional known pharmaceutical excipients and/or additives suitable for use in the formulations of the disclosure are known in the art. e.g., as listed in “Remington: The Science & Practice of Pharmacy”, 21st ed., Lippincolt Williams & Wilkins, (2005), and in the “Physician's Desk Reference”, 60th ed., Medical Economics, Montvale, N.J. (2005). Pharmaceutically acceptable carriers can be routinely selected that are suitable for intravenous administration, solubility and/or stability of the protein as well known in the art or as described herein

It will be understood by one skilled in the art that the liquid pharmaceutical compositions of the disclosure, when formulated as IV admixtures comprising same, may be isotonic with human blood, that is the IV admixture comprising the liquid pharmaceutical composition has essentially the same osmotic pressure as human blood. Such isotonic formulations will generally have an osmotic pressure from about 250 mOSm to about 350 mOSm. Isotonicity can be measured by, for example, using a vapor pressure or ice-freezing type osmometer. Tonicity of a formulation is adjusted by the use of tonicity modifiers. “Tonicity modifiers” are those pharmaceutically acceptable inert substances that can be added to the formulation to provide an isotonicity of the formulation Tonicity modifiers suitable for this disclosure include, but are not limited to, saccharides, salts and amino acids. Concentration of any one or any combination of various components of the liquid pharmaceutical compositions described herein can be adjusted to achieve the desired tonicity of the final IV admixture. For example, the ratio of the carbohydrate excipient to protein may be adjusted according to methods known in the art (e.g., U.S. Pat. No. 6,685,940). The desired isotonicity of the final IV admixture may also be achieved by adjusting the salt concentration of the liquid pharmaceutical composition. Salts that are pharmaceutically acceptable and suitable for this disclosure as tonicity modifiers include, but are not limited to, sodium chloride, sodium succinate, sodium sulfate, potassium chloride, magnesium chloride, magnesium sulfate, and calcium chloride. Amino acids that are pharmaceutically acceptable and suitable as tonicity modifiers include, but are not limited to, proline, alanine, L-arginine, asparagine, L-aspartic acid, glycine, serine, lysine, and histidine.

In some embodiments, liquid pharmaceutical compositions of the disclosure are pyrogen-free, i.e. are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopcial Convention, Pharmacopeial Forum 26 (1):223 (2000)). When therapeutic proteins are administered in amounts of several hundred or thousand milligrams per kilogram body weight, as can be the case with antibodies, even trace amounts of harmful and dangerous endotoxin must be removed. In certain specific embodiments, the endotoxin and pyrogen levels in the composition are less than 10 EU/mg, or less than 5 EU/mg, or less than 1 EU/mg, or less than 0.1 EU/mg, or less than 0.01 EU/mg, or less than 0.001 EU/mg.

The liquid pharmaceutical compositions of the disclosure comprise proteins, such as the therapeutic proteins described herein. In some embodiments, the protein is an antibody. In some embodiments, the protein is a receptor-Fc-fusion protein.

In some embodiments, the liquid pharmaceutical composition comprises 1 to 300 mg/mL of protein, 1 to 250 mg/mL of protein, 1 to 200 mg/mL of protein, 1 to 100 mg/mL of protein, 1 to 50 mg/mL of protein, 1 to 25 mg/mL of protein, 1 to 20 mg/mL of protein, 1 to 10 mg/mL of protein, 1 to 5 mg/mL of protein, 10 to 300 mg/mL of protein, 10 to 250 mg/mL of protein, 10 to 200 mg/mL of protein, 10 to 100 mg/mL of protein, 10 to 50 mg/mL of protein, 10 to 25 mg/mL of protein, 50 to 300 mg/mL of protein, 50 to 250 mg/mL of protein, 50 to 200 mg/mL of protein, 50 to 100 mg/mL of protein, 2 to 25 mg/mL of protein, 2 to 20 mg/mL of protein, 2 to 10 mg/mL of protein, 2 to 5 mg/mL of protein, 5 to 25 mg/mL of protein, 5 to 20 mg/mL of protein, or 5 to 10 mg/mL of protein.

In some embodiments, the liquid pharmaceutical composition is diluted into the IV infusion solution at a ratio of between 1:5 and 1:500, between 1:5 and 1:250, between 1:5 and 1:100, between 1:5 and 1:50, between 1:5 and 1:25, between 1:5 and 1:20, between 1:5 and 1:10, between 1:10 and 1:500, between 1:10 and 1:250, between 1:10 and 1:100, between 1:10 and 1:50, between 1:10 and 1:25, between 1:10 and 1:20, between 1:20 and 1:500, between 1:20 and 1:250, between 1:20 and 1:100, between 1:20 and 1:50, between 1:50 and 1:500, between 1:50 and 1:250, or between 1:50 and 1:100 of liquid pharmaceutical composition to IV infusion solution. The person of ordinary skill in the art will be able to select the appropriate ratio based on protein concentration in the liquid pharmaceutical composition, volume of IV infusion solution, and dose.

It is contemplated that sterile compositions comprising proteins, such as the liquid pharmaceutical compositions described herein, are placed into a container having a sterile access port, for example, an intravenous solution bag or vial having an adapter that allows retrieval of the compositions, such as a stopper pierceable by a hypodermic injection needle. In one embodiment, the compositions are provided in pre-filled syringes. However, any suitable containers are contemplated as within the scope of the instant disclosure.

### Proteins

The disclosure provides liquid pharmaceutical compositions comprising proteins, wherein the liquid pharmaceutical compositions are formulated for delivery of the protein to a subject in an IV admixture.

In some embodiments, the protein comprises a therapeutic protein, i.e. a protein administered to a subject for the treatment of a disease or disorder. Exemplary therapeutic proteins include, but are not limited to antibodies, receptor Fc fusion proteins, such as trap proteins, cytokines, chemokines, growth factors and the like.

In some embodiments, the protein comprises an antigen binding protein, such as an antibody.

The phrase “antigen-binding protein” includes a protein that has at least one complementarity determining region (CDR) and is capable of selectively recognizing an antigen, i.e., is capable of binding an antigen with a KD that is at least in the micromolar range. Therapeutic antigen-binding proteins (e.g., therapeutic antibodies) frequently require a KD that is in the nanomolar or the picomolar range. Typically, an antigen-binding protein includes two or more CDRs, e.g., 2, 3, 4, 5, or 6 CDRs. Examples of antigen binding proteins include antibodies, antigen-binding fragments of antibodies such as polypeptides containing the variable regions of heavy chains and light chains of an antibody (e.g., Fab fragment, F(ab′)2 fragment), and proteins containing the variable regions of heavy chains and light chains of an antibody and containing additional amino acids from the constant regions of heavy and/or light chains (such as one or more constant domains, i.e., one or more of CL, CH1, hinge, CH2, and CH3 domains).

“Antibody” refers to an immunoglobulin molecule consisting of four polypeptide chains, two heavy (H) chains and two light (L) chains inter-connected by disulfide bonds. Each heavy chain has a heavy chain variable region (HCVR or VH) and a heavy chain constant region. The heavy chain constant region contains three domains, CH1, CH2 and CH3. Each light chain has a light chain variable region (VL) and a light chain constant region. The light chain constant region consists of one domain (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from the amino-terminus to the carboxy-terminus in the following order: FR1, CDR1, FR2, CDR2, FR3, CDR3, and FR4. The term “antibody” includes both glycosylated and non-glycosylated immunoglobulins of any isotype or subclass. The term “antibody” includes antibody molecules prepared, expressed, created or isolated by recombinant means, such as antibodies isolated from a host cell transfected with a nucleotide sequence in order to express the antibody. The term “antibody” also includes a bispecific antibody, which includes a heterotetrameric immunoglobulin that can bind to more than one epitope. The term “antibody,” as used herein, also includes antigen-binding fragments of full antibody molecules and fusion proteins comprising antibodies or antigen-binding fragments. A “monoclonal antibody” refers to an antibody that has a specific amino acid sequence, and targets a specific antigen.

The term “antigen-binding portion” of an antibody (or antibody fragment) refers to one or more fragments of an antibody that retain the ability to specifically bind to an antigen. Non-limiting examples of protein binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., Nature (1989) 241:544-546), which consists of a VH domain, (vi) an isolated CDR, and (vii) an scFv, which consists of the two domains of the Fv fragment, VL and VH, joined by a synthetic linker to form a single protein chain in which the VL and VH regions pair to form monovalent molecules. Other forms of single chain antibodies, such as diabodies are also encompassed under the term “antibody”. See, e.g., Holliger et al., PNAS USA (1993) 90:6444-6448; Poljak et al., Structure (1994) 2:1121-1123.

Still further, an antibody or antigen-binding portion thereof may be part of a larger immunoadhesion molecule, formed by covalent or noncovalent association of the antibody or antibody portion with one or more other proteins or peptides. Non-limiting examples of such immunoadhesion molecules include use of the streptavidin core region to make a tetrameric scFv molecule (Kipriyanov et al., Human Antibodies and Hybridomas (1995) 6:93-101) and use of a cysteine residue, a marker peptide and a C-terminal polyhistidine tag to make bivalent and biotinylated scFv molecules (Kipriyanov et al. Mol. Immunol. (1994) 31:1047-1058). Antibody portions, such as Fab and F(ab′)2 fragments, can be prepared from whole antibodies using conventional techniques, such as via papain or pepsin digestion of whole antibodies. Moreover, antibodies, antibody portions and immunoadhesion molecules can be obtained using standard recombinant DNA techniques commonly known in the art (see Sambrook et al., 1989).

The term “human antibody” is intended to include antibodies having variable and constant regions derived from human germline immunoglobulin sequences. Human antibodies of the present disclosure may include amino acid residues not encoded by human germline immunoglobulin sequences (e.g., mutations introduced by random or site-specific mutagenesis in vitro or by somatic mutation in vivo), for example in the CDRs and in particular CDR3.

The term “recombinant human antibody”, as used herein, is intended to include all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see, e.g., Taylor et al. Nucl. Acids Res. (1992) 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis), and thus the amino acid sequences of the VH and VL regions of the recombinant antibodies are sequences that, while derived from and related to human germline VH and VL sequences, may not naturally exist within the human antibody germline repertoire in vivo.

Additional therapeutic proteins are contemplated as within the scope of the instantly disclosed methods of cell culture and therapeutic protein production. In certain embodiments, the therapeutic protein is an antibody, a human antibody, a humanized antibody, a chimeric antibody, a monoclonal antibody, a multispecific antibody, a bispecific antibody, an antigen binding antibody fragment, a single chain antibody, a diabody, triabody or tetrabody, a Fab fragment or a F(ab′)2 fragment, an IgD antibody, an IgE antibody, an IgM antibody, an IgG antibody, an IgG1 antibody, an IgG2 antibody, an IgG3 antibody, or an IgG4 antibody. In certain embodiments, the antibody is an IgG1 antibody, an IgG2 antibody, an IgG4 antibody, a chimeric IgG2/IgG4 antibody, a chimeric IgG2/IgG1 antibody or a chimeric IgG2/IgG1/IgG4 antibody.

In some embodiments, the antibody is selected from the group consisting of an anti-Programmed Cell Death 1 antibody (e.g., an anti-PD1 antibody as described in U.S. Pat. Appln. Pub. No. US2015/0203579A1), an anti-Programmed Cell Death Ligand-1 (e.g., an anti-PD-L1 antibody as described in U.S. Pat. Appln. Pub. No. US2015/0203580A1), an anti-D114 antibody, an anti-Angiopoctin-2 antibody (e.g., an anti-ANG2 antibody as described in U.S. Pat. No. 9,402,898), an anti-Angiopoetin-Like 3 antibody (e.g., an anti-AngPt13 antibody as described in U.S. Pat. No. 9,018,356), an anti-platelet derived growth factor receptor antibody (e.g., an anti-PDGFR antibody as described in U.S. Pat. No. 9,265,827), an anti-Erb3 antibody, an anti-Prolactin Receptor antibody (e.g., anti-PRLR antibody as described in U.S. Pat. No. 9,302,015), an anti-Complement 5 antibody (e.g., an anti-C5 antibody as described in U.S. Pat. Appln. Pub. No US2015/0313194A1), an anti-TNF antibody, an anti-epidermal growth factor receptor antibody (e.g., an anti-EGFR antibody as described in U.S. Pat. No. 9,132,192 or an anti-EGFRvIII antibody as described in U.S. Pat. Appln. Pub. No. US2015/0259423A1), an anti-Proprotein Convertase Subtilisin Kexin-9 antibody (e.g. an anti-PCSK9 antibody as described in U.S. Pat. No. 8,062,640 or U.S. Pat. Appln. Pub. No. US2014/0044730A1), an anti-Growth And Differentiation Factor-8 antibody (e.g., an anti-GDF8 antibody, also known as anti-myostatin antibody, as described in U.S. Pat. No. 8,871,209 or U.S. Pat. No. 9,260,515), an anti-Glucagon Receptor (e.g., anti-GCGR antibody as described in U.S. Pat. Appln. Pub. Nos. US2015/0337045A1 or US2016/0075778A1), an anti-VEGF antibody, an anti-IL1R antibody, an interleukin 4 receptor antibody (e.g., an anti-IL4R antibody as described in U.S. Pat. Appln. Pub. No. US2014/0271681A1 or U.S. Pat. No. 8,735,095 or U.S. Pat. No. 8,945,559), an anti-interleukin 6 receptor antibody (e.g., an anti-IL6R antibody, as described in U.S. Pat. Nos. 7,582,298, 8,043,617 or U.S. Pat. No. 9,173,880), an anti-IL1 antibody, an anti-IL2 antibody, an anti-IL3 antibody, an anti-IL4 antibody, an anti-IL5 antibody, an anti-IL6 antibody, an anti-IL7 antibody, an anti-interleukin 33 (e.g., anti-IL33 antibody as described in U.S. Pat. Appln. Pub. Nos. US2014/0271658A1 or US2014/0271642A1), an anti-Respiratory syncytial virus antibody (e.g., anti-RSV antibody as described in U.S. Pat. Appln. Pub. No. US2014/0271653A1), an anti-Cluster of differentiation 3 (e.g., an anti-CD3 antibody, as described in U.S. Pat. Appln. Pub. Nos. US2014/0088295A1 and US20150266966A1, and in U.S. Application No. 62/222,605), an anti-Cluster of differentiation 20 (e.g., an anti-CD20 antibody as described in U.S. Pat. Appln. Pub. Nos. US2014/0088295A1 and US20150266966A1, and in U.S. Pat. No. 7,879,984), an anti-CD19 antibody, an anti-CD28 antibody, an anti-Cluster of Differentiation-48 (e.g., anti-CD48 antibody as described in U.S. Pat. No. 9,228,014), an anti-Fel d1 antibody (e.g., as described in U.S. Pat. No. 9,079,948), an anti-Middle East Respiratory Syndrome virus (e.g., an anti-MERS antibody as described in U.S. Pat. Appln. Pub. No. US2015/0337029A1), an anti-Ebola virus antibody (e.g., as described in U.S. Pat. Appln. Pub. No. US2016/0215040), an anti-Zika virus antibody, an anti-Lymphocyte Activation Gene 3 antibody (e.g., an anti-LAG3 antibody, or an anti-CD223 antibody), an anti-Nerve Growth Factor antibody (e.g., an anti-NGF antibody, as described in U.S. Pat. Appln. Pub. No. US2016/0017029 and U.S. Pat. Nos. 8,309,088 and 9,353,176) and an anti-Activin A antibody. In some embodiments, the bispecific antibody is selected from the group consisting of an anti-CD3×anti-CD20 bispecific antibody (as described in U.S. Pat. Appln. Pub. Nos. US2014/0088295A1 and US20150266966A1), an anti-CD3×anti-Mucin 16 bispecific antibody (e.g., an anti-CD3×anti-Muc16 bispecific antibody), an anti-CD3×anti-Prostate-specific membrane antigen bispecific antibody (e.g., an anti-CD3×anti-PSMA bispecific antibody), an anti-severe acute respiratory syndrome coronavirus 2 (SARS-COV-2) antibody, an anti-MET proto-oncogene, receptor tyrosine kinase (MET) antibody, an anti-BCMA×anti-CD3 bispecific antibody, an anti-interleukin 4 receptor (IL-4R) antibody, a leptin receptor (LEPR) agonist antibody, an anti-sigma factor NepR (NEPR1) agonist antibody, an anti-interleukin 6 receptor (IL-6R) antibody, an anti-interleukin 33 (IL-33) antibody and an anti-Bet v 1 antibody. In some embodiments, the protein is selected from the group consisting of atoltivimab, maftivimab, odesivimab, odronextamab, ubamatamab, cemiplimab, garetosmab, odronextamab, pozelimab, mibavademab, fianlimab, itepekimab, alirocumab, sarilumab, fasinumab, nesvacumab, dupilumab, trevogrumab, evinacumab, and rinucumab. In some embodiments, the protein is selected from the group consisting of alirocumab, sarilumab, fasinumab, nesvacumab, dupilumab, trevogrumab, evinacumab, and rinucumab.

In other embodiments, the protein is a recombinant protein that contains an Fc moiety and another domain, (e.g., an Fc-fusion protein). In some embodiments, an Fc-fusion protein is a receptor Fc-fusion protein, which contains one or more extracellular domain(s) of a receptor coupled to an Fc moiety. In some embodiments, the Fc moiety comprises a hinge region followed by a CH2 and CH3 domain of an IgG. In some embodiments, the receptor Fc-fusion protein contains two or more distinct receptor chains that bind to either a single ligand or multiple ligands. For example, an Fc-fusion protein is a trap protein, such as for example an IL-1 trap (e.g., rilonacept, which contains the IL-IRAcP ligand binding region fused to the Il-1R1 extracellular region fused to Fc of hIgG1; see U.S. Pat. No. 6,927,004, which is herein incorporated by reference in its entirety), a VEGF trap (e.g., aflibercept or ziv-aflibercept, which contains the Ig domain 2 of the VEGF receptor Flt1 fused to the Ig domain 3 of the VEGF receptor Flk1 fused to Fc of hIgG1; see U.S. Pat. Nos. 7,087,411 and 7,279,159; or conbercept, which contains the Ig domain 2 of the VEGF receptor Flt1 fused to the Ig domain 3 of the VEGF receptor Flk1 fused to the Ig domain 4 of the VEGF receptor Flk1 fused to Fc of hIgG1; see U.S. Pat. No. 8,216,575), or a TNF trap (e.g., etanercept, which contains the TNF receptor fused to Fc of hIgG1; see U.S. Pat. No. 5,610,279). In other embodiments, an Fc-fusion protein is a ScFv-Fc-fusion protein, which contains one or more of one or more antigen-binding domain(s), such as a variable heavy chain fragment and a variable light chain fragment, of an antibody coupled to an Fc moiety.

In some embodiments, the protein is a glycoprotein. Glycoproteins with asparagine-linked (N-linked) glycans are ubiquitous in eukaryotic cells. Biosynthesis of these glycans and their transfer to polypeptides takes place in the endoplasmic reticulum (ER). N-glycan structures are further modified by a number of glycosidases and glycosyl-transferases in the ER and the Golgi complex. Glycosylation of therapeutic proteins can be critical for therapeutic protein quality and effectiveness. For example, antibody glycosylation is a common post-translational modification, and may play a role in antibody effector function, as well as antibody stability. Methods of analyzing glycosylation patterns, and the percentage glycosylated protein in a sample of protein, will be known to the skilled artisan.

The following Examples are merely illustrative and are not meant to limit any aspects of the present disclosure in any way.

### EXAMPLES

### Example 1: Methods for Examples 2-5

### Reagents and Materials

A representative monoclonal antibody, monoclonal antibody 1 (mAb1) without polysorbate 20 was produced in house according to standard methods, and was used for all experiments described below. The antibody was formulated to 60 mg/mL (low concentration) or 200 mg/mL (high concentration) in histidine pH 6.3. Super-refined polysorbate 20 was purchased from J. T. Baker (Phillipsburg, NJ). 10% (w/v) polysorbate 20 was spiked into mAb1 at varying concentrations to produce mAb1+polysorbate 20. Diluent Polyvinyl chloride (PVC) IV bags were purchased from Baxter International (Deerfield, IL), Hospira (Lake Forest, IL), and B. Braun Medical Inc. (Bethlehem, PA) containing 50 or 100 mL of diluent and were used for IV admixtures (surface area to volume ratios were 2.7-3.9 for 50 mL bags and 1.7-2.5 for 100 mL IV bags). PVC with di-(2-ethylhexyl) phthalate (DEHP) IV infusion sets were purchased from BD (Franklin Lakes, NJ) and Baxter International (Deerfield, IL). 20 or 22 G catheters and 0.2 μm polyethersulfone (PES) filters were purchased from BD (Franklin Lakes, NJ) and Pall (Washington, NY), respectively, and were attached to the infusion sets. The Gemini PC-1® pump was purchased from BD (Franklin Lakes, NJ) and the FloGard® 6201 was purchased from Baxter International (Deerfield, IL) and were used to deliver the contents from the IV bags.

Preparation of mAb1 Plus Polysorbate 20 Formulations

Several mAb1 plus polysorbate 20 formulations were prepared ranging from 0% to 60% of the polysorbate 20 levels found in the DP formulation. Polysorbate 20 ranging from 0% to 0.03% (w/v) was added. These levels were chosen to represent polysorbate degradation in the DP formulation nominally containing 0.05% (w/v) polysorbate 20. In all cases, the mAb1+polysorbate 20 formulations were filtered with a 0.22 μm polyvinylidene fluoride (PVDF) filter.

### Polysorbate 20 Quantitation

The concentration of polysorbate 20 in the mAb1plus polysorbate 20 formulations was determined by ultra-performance liquid chromatography (UPLC) with a charge aerosol detector (CAD). Quantitation was performed using an Acquity H-Class UPLC system purchased from Waters (Milford, MA) with a Corona® Ultra/Veo RS™ Charged Aerosol Detector purchased from Thermo Fisher Scientific (Waltham, MA), and an Oasis Max 30 μm, 2.1×20 mm column purchased from Waters™ (Milford, MA). Mobile phase A (MPA) consisted of 0.1% formic acid (FA) in water, and mobile phase B (MPB) consisted of 0.1% FA in acetonitrile used in a step gradient at 1.0 mL/min for 8 minutes. The initial ratio of 90% MPA/10% MPB changed to 80% MPA/20% MPB at 1.0 minute and continued as such until 3.4 minutes. From 3.5 minutes to 4.5 minutes, 100% MPB was used. From 4.6 minutes through 8 minutes, the gradient returned to the initial ratio of 90% MPA/10% MPB. A standard curve created with a known polysorbate 20 reference standard was used to quantify the amount of polysorbate 20 present in the test articles. Standards and samples were injected at volumes between 0.3 and 3.0 μL, with protein samples targeting a column load of 0.21 μg. Data analysis was performed with Waters Empower™ 3 Feature Release 5.

A mAb1 reference standard containing a known concentration of mAb1 and polysorbate 20 was run with all mAb1 plus polysorbate 20 formulations at a column load of 0.21 μg polysorbate 20, and the known polysorbate 20 concentration from that lot was used to normalize the concentration of mAb1 plus polysorbate 20 formulations from different sequences.

### IV Admixture Dose Preparation

IV admixtures were prepared in both 50 mL and 100 mL IV bags. Due to the overfill associated with the IV bags, the final polysorbate concentration was measured, and the measured concentration was used. A volume of diluent equal to the volume of mAb1 plus polysorbate 20 added to the IV bag was removed prior to the addition of mAb1+polysorbate 20. After addition of mAb1 plus polysorbate 20, each IV bag was mixed thoroughly. The target protein concentration in the IV admixture of 0.85 mg/mL mAb1 in 100 mL IV bags and 1.6 mg/mL in 50 mL IV bags was verified by RP-UPLC. All measured protein concentrations were within 5% of the target values. The IV bags were incubated for 24 hours at 2-8° C., and then for at least at an additional 8 hours at 25° C. After storage, an infusion set was connected to each IV bag with a catheter and filter, and then primed by gravity and held at ambient room temperature for 60 minutes. The IV bags were then connected to IV pumps and delivered into a rinsed and cleaned polystyrene container at rates between 25 and 100 mL/hr (50 mL/hr for IV admixtures prepared in 50 mL IV bags, and 100 mL/hr for IV admixtures prepared in 100 mL IV bags).

### Visual Inspection

Visible particles were examined without any magnification against a black background and a white background while using ambient lighting. The samples were then gently swirled and inspected against a StablCal Reference Suspension Set purchased from Hach (Loveland, CO). Color was assessed with Color Reference Solutions BY according to Ph. Eur. purchased from Sigma-Aldrich (St. Louis, MO).

### Size Exclusion-Ultra Performance Liquid Chromatography (SE-UPLC)

The purity of the mAb1 plus polysorbate 20 formulations was determined by SE-UPLC with an Acquity UPLC H-Class system and an ACQUITY UPLC BEH200 SEC 1.7 μm, 4.6×300 mm column purchased from Waters (Milford, MA). The mobile phase consisted of 10 mM sodium phosphate and 1M sodium perchlorate. Test articles were injected isocratically at 0.3 mL/min for 15 minutes and the absorbance of the samples measured at 280 nm. The percentage of each protein species was determined by the relative abundance and area under the peak.

### Sub-Visible Particle Analysis by Light Obscuration

Particle size was determined by light obscuration with a HIAC 9703+ Liquid Particle Counting System equipped with a HRLD 400 CE/Standard Sensor with a theoretical size range of 2-400 μm, and a Tecan 1 mL Sample Syringe obtained from Beckman Coulter Life Sciences (Indianapolis, IN). Instrument performance was verified using 15 μm polystyrene count and size standards purchased from Thermo Fisher Scientific (Waltham, MA). Before particle analysis, samples were vacuum degassed for 15 minutes. Subvisible particles were reported for particles with ≥10 μm and >25 μm, as of the indicated size particles per container. To measure particle counts, four 1 mL aliquots were drawn from the samples, and the average particle count of the last three draws was reported.

### Sub-Visible Particle Analysis by Flow Imaging

Particles were further characterized by MFI™ 5200 flow microscopy with a Bot1 Autosampler equipped with a 100 μm flow cell, 1.6 mm, with silane coating (Bio-Techne. Minneapolis, MN; Protein Simple, Inc., Santa Clara, CA). The peristaltic pump was calibrated using water. The flow cell was focused using a 10 μm Duke Standard™ size standard, and system suitability was ensured with a COUNT-CAL™ 3000/mL, 5 μm concentration standard, both purchased from Thermo Fisher Scientific (Waltham, MA). Following a manual prime of the flow cell with water, 0.86 mL of sample volume was drawn from the sample plate. 0.15 mL was used to flush the flow cell, and 0.1 mL was used to optimize the illumination and subtract the flow cell background. 0.6 mL of each sample was then analyzed. MFI Image Analysis (software version 1.1) was used with remove stuck and remove edge filters to view images of the particles, and the number of particles per container for 2-10 μm, ≥10 μm, and >25 μm were reported. Results are reported in maximum feret diameter, which is defined as a measure of diameter expressed in microns along the longest axis.

### Raman Microscopy

Samples from mAb1 studies showing increased particle formation over time were analyzed with a Single Particle Explorer purchased from rap.ID (Monmouth Junction, NJ) to identify the particles seen with an internal identification database. A Lambda wavelength calibration (520 cm-1) and Raman spectroscopy calibration were performed prior to sample analysis. Samples were prepared with 5 μm gold-coated polycarbonate filters and a glass filter funnel, where 100 μL of sample was pipetted onto the filter with a clean pipette tip and rinsed with cold water at >10-fold the sample volume. The filtered sample was vacuum dried before analysis.

### Curve Fitting and Analysis

Polysorbate 20 degradation kinetics were fit to an exponential decay model shown in Equation 1:

Polysorbate 20,%(w/v)=a*Exp(b*time, months) (Equation 1)

In the model, a is the scale, and b is the growth rate. “Exp” stands for “Exponential” or “e.” The data were fit to the exponential decay model using nonlinear regression modeling in JMP16 (JMP Statistical Discovery, LLC).

### Protein Quantitation by Reverse Phase-Ultra Performance Liquid Chromatography (RP-UPLC)

The protein concentration in IV admixtures was determined by RP-UPLC with an Acquity UPLC H-Class system purchased from Waters (Milford, MA) and a Zorbax 300SB-CN Rapid Resolution, 4.6×50 mm column purchased from Agilent (Santa Clara, CA). Mobile phase A (MPA) consisted of 0.1% TFA in high purity water and mobile phase B (MPB) consisted of 0.1% TFA in acetonitrile. 3.0 μg of sample were injected at 1.0 mL/min along a gradient with initial conditions of 90% MPA/10% MPB. The gradient changed to 10% MPA/90% MPB for 1.4 minutes before returning to the initial conditions. A standard curve was created with a mAb1 reference standard. Data analysis was performed with Waters Empower 3 Feature Release 5.

### Example 2: Identification of Polysorbate (PS) 20 Degradation in an Antibody Drug Product

mAb1 has the potential to be delivered as a both high-concentration subcutaneous formulation and a lower-concentration IV liquid formulation. Accordingly, mAb1 formulations were developed along two parallel paths, a low concentration formulation and a high concentration formulation, with both of these delivery methods in mind. The excipient composition for both the subcutaneous and IV formulations were identical. The only difference between the two formulations was the concentration of mAb1. Both formulations contained the same concentration of polysorbate 20. Both formulations were stored in glass vials in the upright orientation, and were stored at 5° C. for up to 36 months.

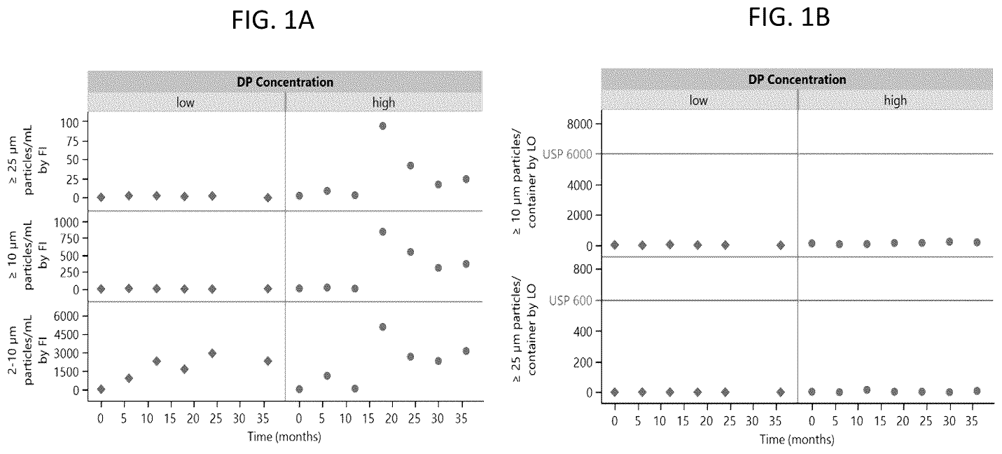

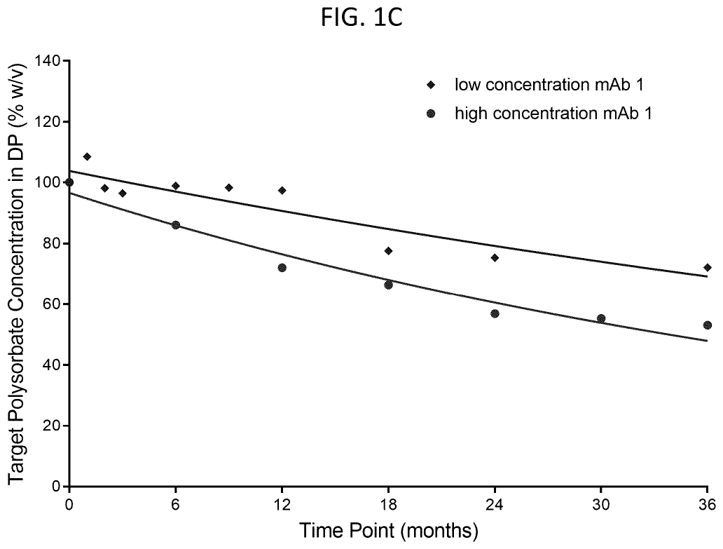

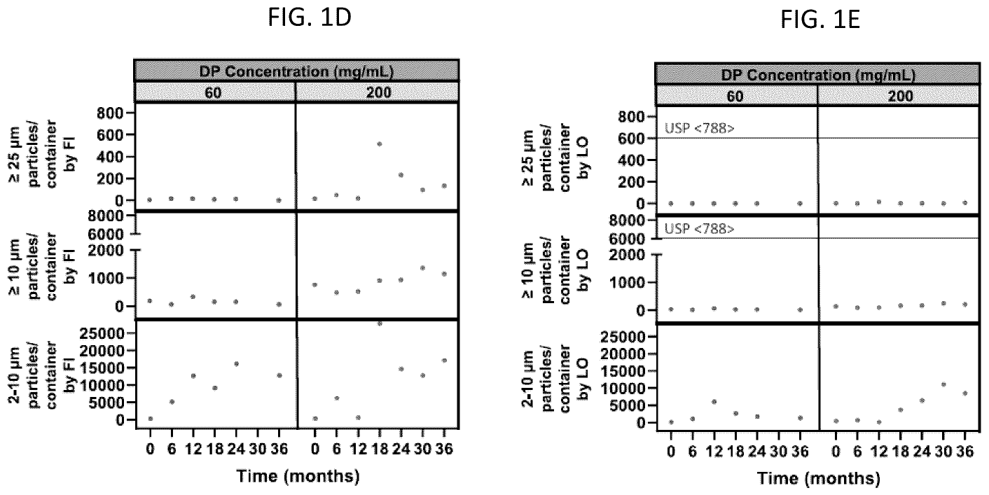

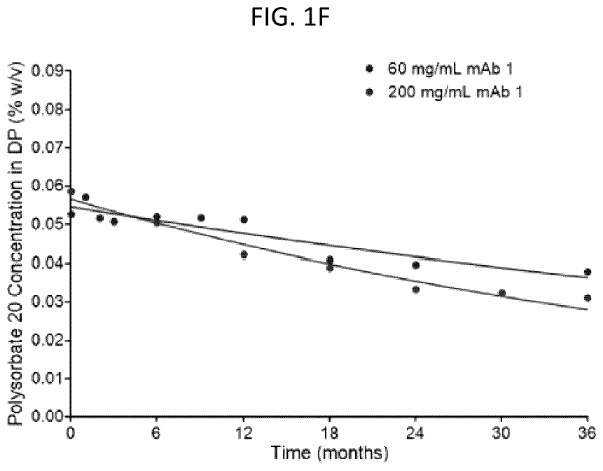

Particle analysis is a standard assay, and was included and monitored by FI and LO. Both the high and low mAb1 concentration formulations were subjected to particle analysis by FI and LO, as described in Example 1. Particles were observed by FI during storage at 5° C. A notable increase in 2-10 μm particles, ≥10 μm particles, and >25 μm particles was observed by FI after storage of the high-concentration formulation at 5° C. for 18 months (FIGS. 1A and 1D, right side). The low-concentration formulation (FIGS. 1A and 1D, left side) did not show a similar dramatic spike in particles. However, the low-concentration formulation did show a steady increase in 2-10 μm particles during storage at 5° C. (FIGS. 1A and 1D, left side). Particles increased consistently up to 24 months and then leveled off. The trend in increasing particles was evident by FI, but was not observed by LO when assessing particles with sizes ≥10 μm (FIGS. 1B and 1E, top) or greater than ≥25 μm (FIGS. 1B and 1E, bottom). No meaningful increases in particles were observed by LO, and all particle concentrations were well within the limits specified in USP <788> (FIGS. 1B and 1E). Thus, with respect to particle concentrations, the quality of the vialed drug product of mAb1 at both the low and high concentration formulations is considered acceptable, and within the limits considered safe.

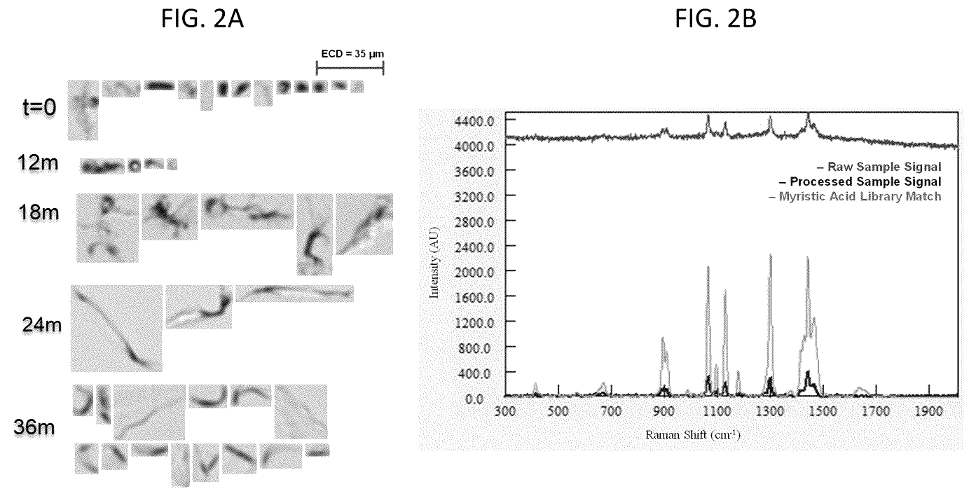

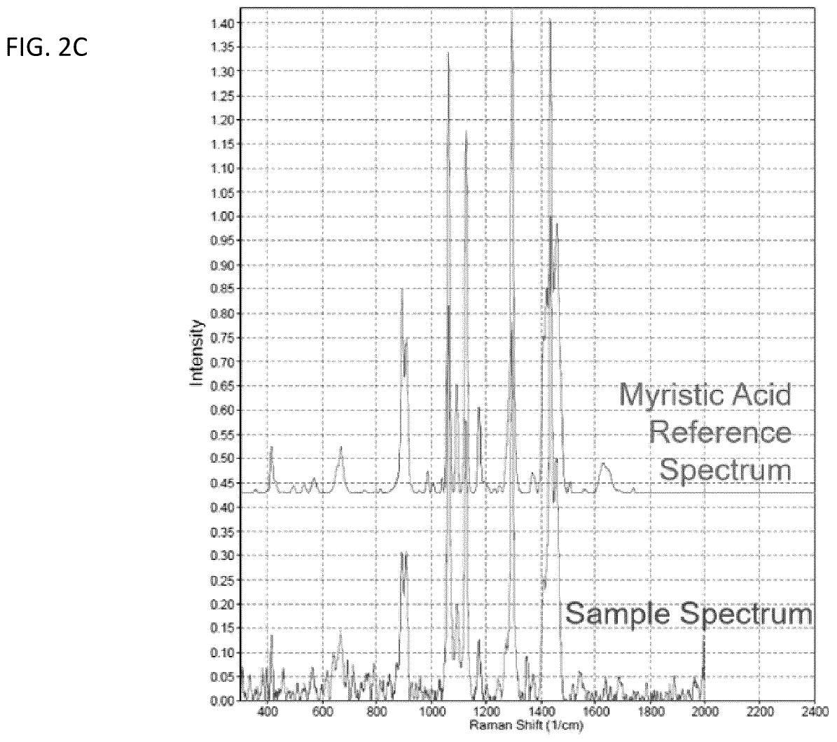

The observation of increasing particle concentrations by FI prompted an investigation to further understand the nature of the particles and the root cause for their formation. Analysis of the images from FI indicated that the particles that formed over time were fibrous (FIG. 2A). Further analysis by Raman microscopy indicated that the chemical nature of the particles was consistent with fatty acids such as myristic acid and lauric acid (FIGS. 2B-2C). The instrument software provided a best match of the sample spectrum to a reference spectrum of myristic acid (FIGS. 2B-2C). Both myristic acid and lauric acid resulted in good fit scores of 966 and 948 (out of 1000), respectively, and was interpreted as a mixture of fatty acids.

The samples were assessed for polysorbate 20 levels over time by CAD-UPLC, and the data were fit to an exponential decay. These results indicated that there was observable polysorbate degradation occurring in a DP lot manufactured from a research lot of drug substance (FIG. 1C, reported as the actual amount in the solution, relative to the target concentration, and FIG. 1F, reported as polysorbate concentration by % w/v). In the high-concentration formulation, polysorbate 20 degraded by approximately 50% over the course of 36 months of storage at 5° C. and in the low concentration formulation, the degradation was approximately 30% relative to initial levels. This suggested that the degradation was related to the concentration of mAb1, since all other formulation components were the same between the two formulations. One possibility was that the degradation of polysorbate 20 was not due to auto-oxidation, but rather due to enzymatic hydrolysis due to the presence of host-cell lipases. The presence of host-cell lipases was in fact confirmed and shown to be due to two specific lipases, lysosomal acid lipase and lipoprotein lipase. Hydrolytic polysorbate degradation is known to be negligible under the fairly neutral pH 6.3 storage condition, with the excipients used, and during the 5° C. storage temperature tested. With the exception of the presence of the host-cell lipases, no additional quality attributes were impacted, as described in Example 5 below. Size exclusion-UPLC showed no increases in aggregate species. Capillary electrophoresis showed no changes in low molecular weight species. No changes in the distribution of charge variants were observed. Potency by cell-based bioassay was maintained over the 36 month storage period at 5° C.

### Example 3: In-Use Stability of mAb1 in the Presence and Absence of Polysorbate 20 in IV Admixtures

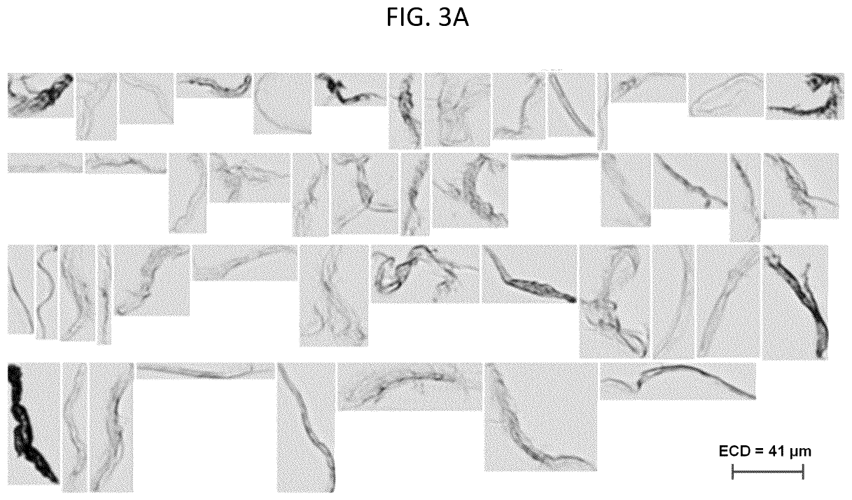

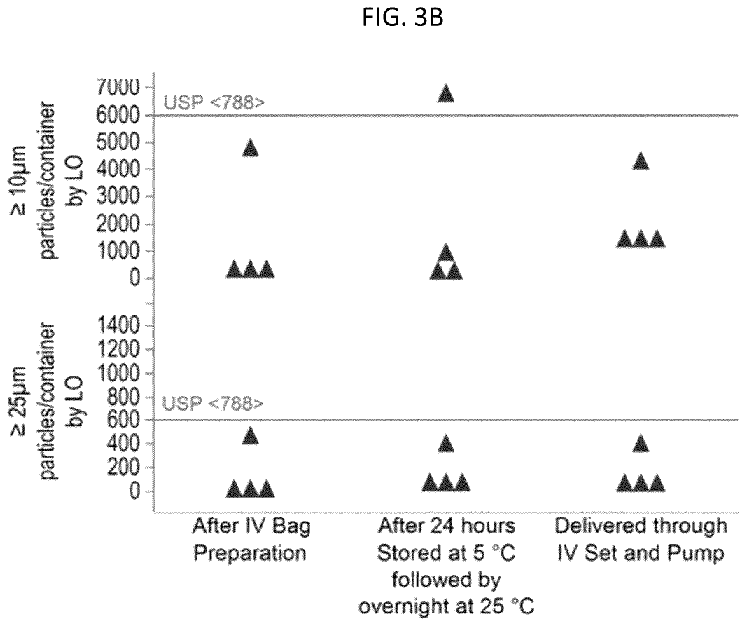

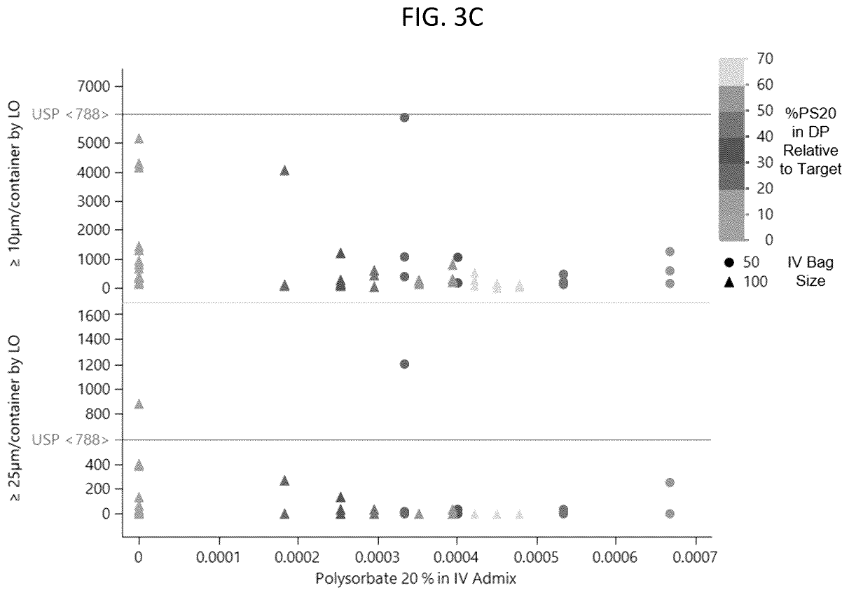

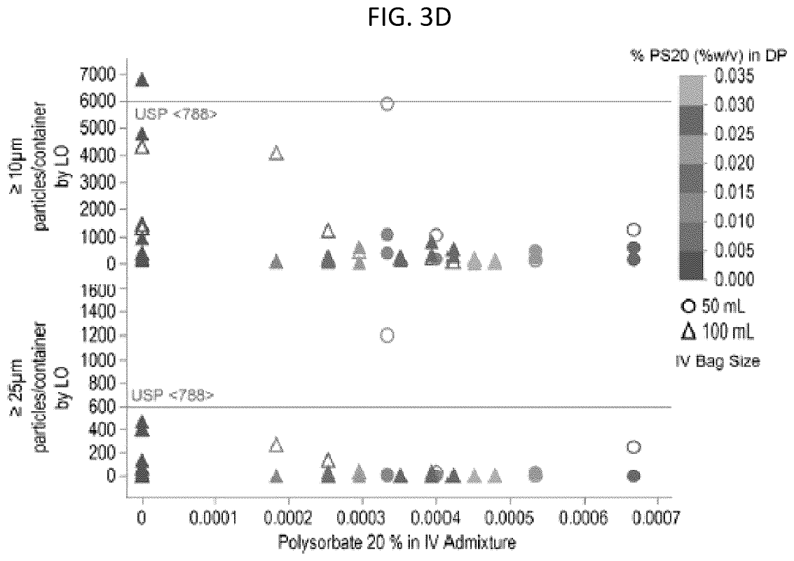

Polysorbate 20 is required to stabilize mAb1 to interfacial and handling stresses during manufacturing and handling during product life cycle. It is also required to stabilize the mAb1 IV admixture to interfacial stresses encountered during preparation and administration (including hold times, mixing, and dilution with diluent) and to reduce antibody adsorption to the surfaces of materials used in the delivery device. Since polysorbate 20 degradation was observed in mAb1 DP samples stored at 5° C., we sought to understand the limit to which polysorbate 20 degradation in mAb1 DP can be tolerated during IV administration by initiating a study to assess the impact of having no polysorbate 20 in IV admixtures. As such, IV admixtures were prepared from mAb1 DP formulated without polysorbate 20. After storage or after delivery of the IV admixture, sub-visible particles were observed, and the morphology of the sub-visible particles was observed by FI to be large and fibrous in nature (FIG. 3A). As no polysorbate 20 was present in this formulation, fatty acid particles were ruled out as the nature of these particles. The observed particles are consistent with the morphology of protein aggregates. Additionally, in one scenario tested for this study, the level of particles exceeded the limits set forth in USP <788> (see FIG. 3B). The protein recovery of all samples tested was within 5% of the starting concentration. Based on these observations, we determined that polysorbate 20 was required to stabilize the IV admixture to the formation of particles. Without wishing to be bound by theory, we hypothesized that a minimum level of polysorbate 20 was required in the IV admixture and designed a testing strategy to determine this.

### Example 4: Simulation of Polysorbate 20 Degradation During Shelf-Life and its Effect on IV Admixtures

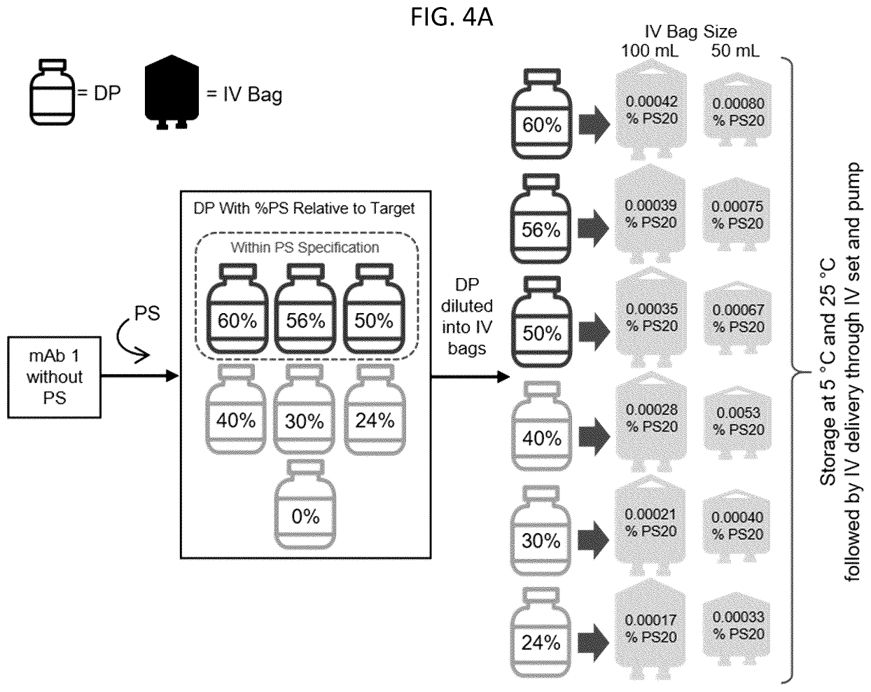

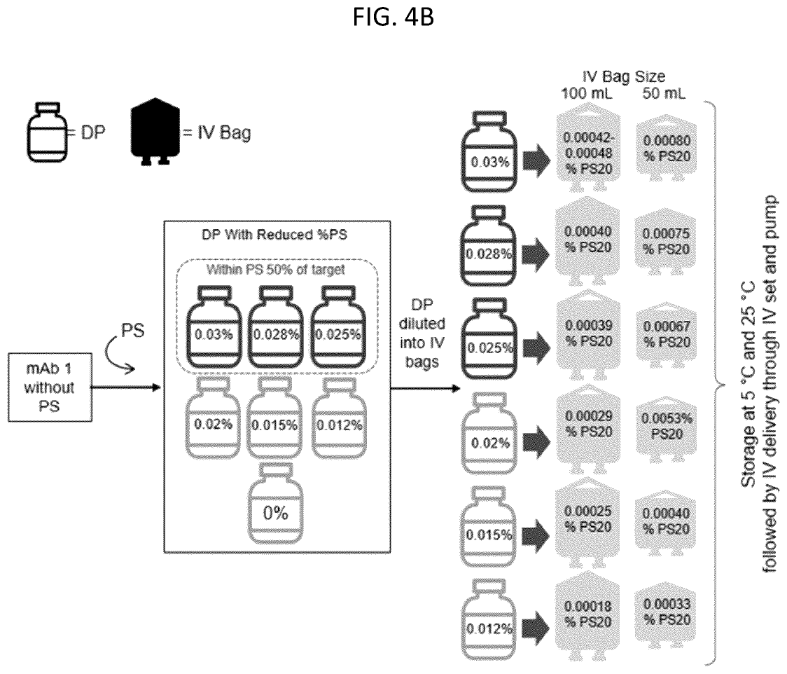

An experiment to simulate the levels of polysorbate 20 that might be found in an antibody drug product over the course of its shelf-life, if host-cell lipases were present, was developed. The goal was to determine if the initial levels of polysorbate 20 included in the product were sufficient to ensure not only the quality of the drug product, but also the quality of the IV admixture prepared from a drug product containing trace amounts of host-cell lipases. Bulk formulated drug substance was prepared with all formulation components at target levels, but without any polysorbate 20. Different levels of polysorbate 20 were spiked in to simulate the levels that might be found in the drug product over the course of the shelf-life if the polysorbate was being degraded by host-cell lipases. The study design is illustrated in FIGS. 4A-4B.

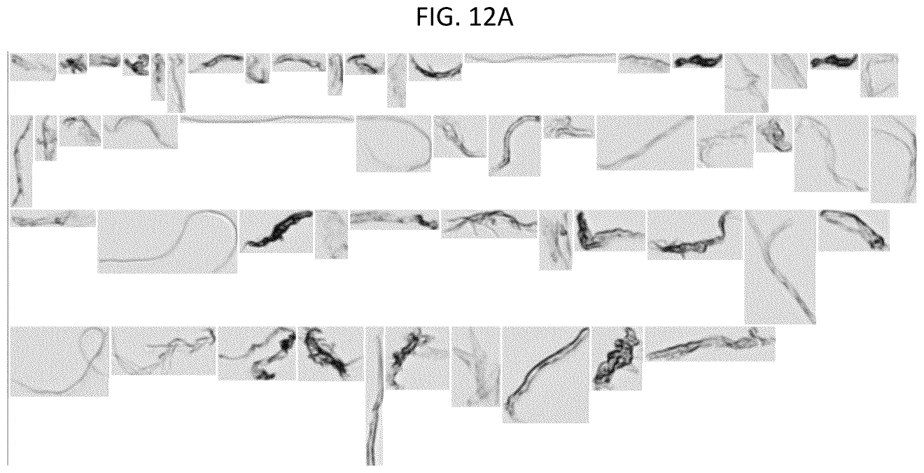

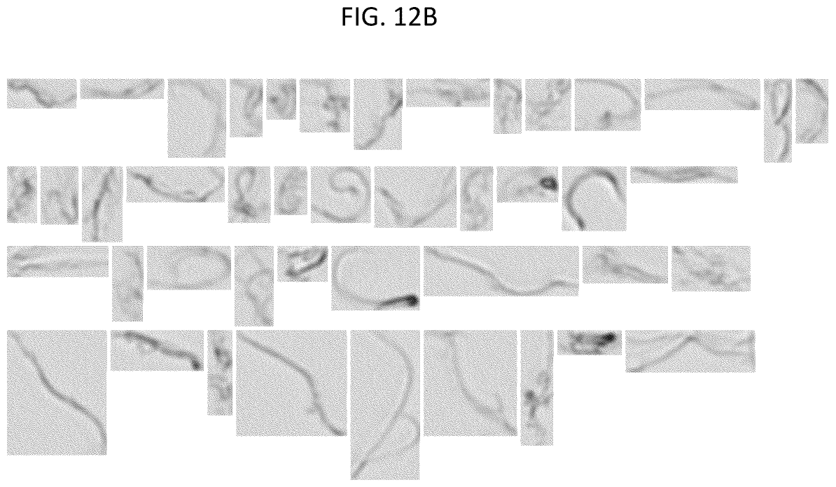

An increasing trend in subvisible particles was observed by LO (particles both ≥10 μm and ≥25 μm) as the levels of polysorbate 20 in the IV admixture decreased (FIGS. 3C-3D). The trend in particle levels in the sample delivered through the IV infusion set remained stable when the polysorbate 20 concentration was approximately 0.0004% or greater (in units of w/v). When the polysorbate 20 concentration in the admixture decreased below 0.0004%, the level of particles in the solution delivered through the infusion set increased. In two cases, the level of particles exceeded the limits set forth in USP <788>. The first case was when 0% (w/v) polysorbate was included in the formulation. The other case that failed USP <788> (0.00033% (w/v) polysorbate 20) had more darker particles, compare to some other samples, and these could be distinguished by LO (FIGS. 12A-12B). This sample was delivered through the infusion set and had similar particle counts as other IV admixtures delivered with less than 0.0004% (w/v) polysorbate as seen by FI (Table 4-1, below). This shows the importance of using FI as an orthogonal method to LO for particle analysis. In most cases, increased particle counts were seen for the delivered IV admixture and not the in the initial (t=0_sample or the IV admixture storage sample, suggesting that stressed encountered from the infusion process or interactions with the materials of the infusion set contributed to instability in the IV admixture when polysorbate concentration was lower than 0.0004% (w/v) in the IV admixture.

These results indicate that the minimum polysorbate 20 level required to stabilize the IV admixture should be conservatively set at 0.0004% (w/v) to guarantee patient safety. Final IV bag volume did not seem to impact the level of particles. Regardless of the final volume in the IV bag, the trend in increasing particles held when crossing the threshold value of 0.0004% (w/v). This suggests that the resulting instability is not a function of surface area to volume ratio within the range tested. Particle formation does not appear to be a function of IV bag or infusion set material, when sufficient polysorbate 20 is present. Although this study reports results from a limited set of materials, previous development studies performed with a broad range of materials, including IV bags made from PVC or polyolefin, and IV infusion sets made from polyurethane, PVC containing DEHP, PVC containing TOTM and polyethylene lined PVC, indicated that IV bag and infusion set materials did not impact the in-use stability or quality of IV admixtures of this mAb (see Table 4-2 and 4-3 below). Furthermore, flow rate did not impact the in-use stability of IV admixtures. Flow rate was varied between 25 and 500 mL/hour in previous IV studies with mAb1 and was determined not to impact particulate matter formation (tables 4-2 and 4-3).

LO provides a quantitative measurement of the number and size of particles in solution, which can be used to assess whether injectable products meet the USP chapter USP <788> provides guidance on the amount of acceptable particulate matter, which is a benchmark many pharmaceutical companies use for IV admixtures. However, LO fails to provide any information on the morphology of particles. Understanding particle morphology can help assess the mechanism for any observed instabilities associated with parenteral products. Flow imaging (FI) is a complementary technique to LO, in that it provides not only particle size and concentration, but also yields images of individual particles that can aid in understanding the nature of the particles. The same samples analyzed by LO were analyzed by FI. Images of particles obtained from FI indicated that the morphology of the particles was fibrous and consistent with proteinaceous particles (FIG. 3A). Therefore, the maximum Feret diameter was chosen as a parameter to characterize the trend in particle formation.

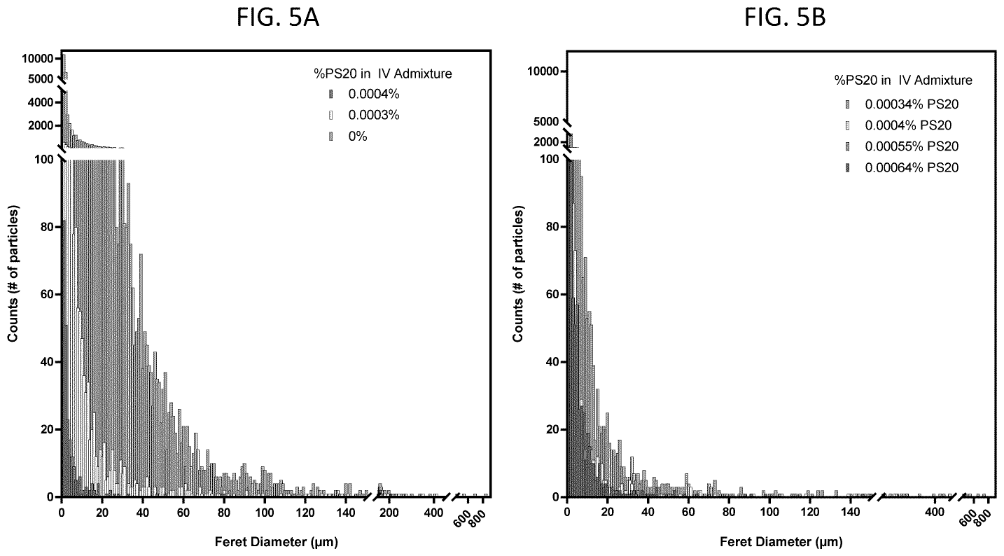

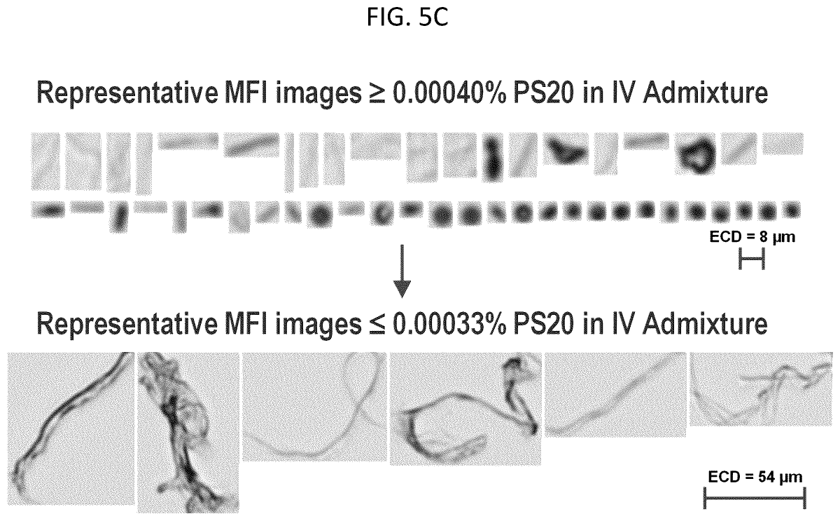

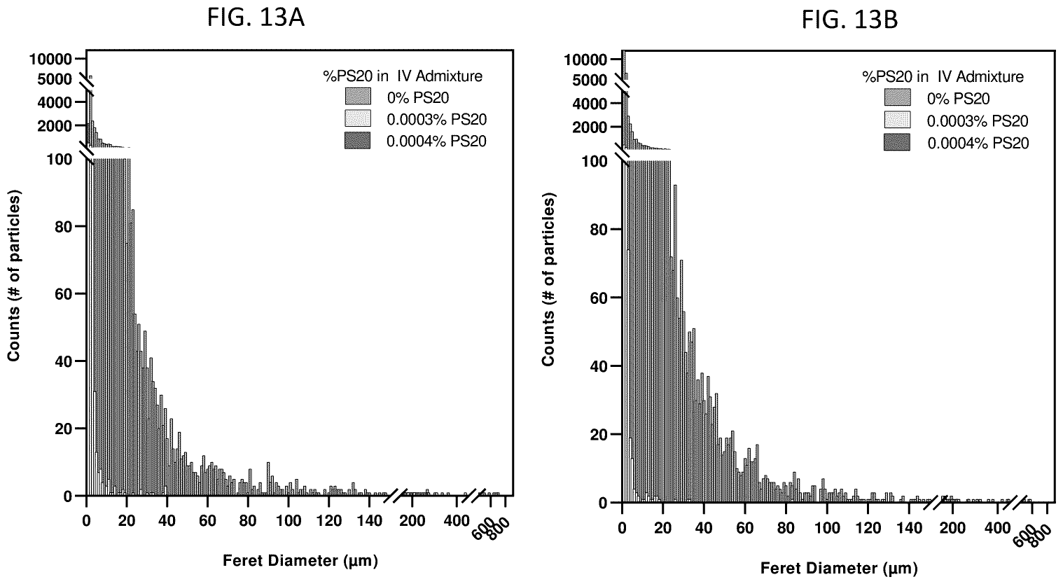

FIGS. 5A and 5B show the distribution of particle counts vs. maximum Feret diameter for delivered mAb1 admixtures prepared with decreasing concentrations of polysorbate 20, as described above. Two sets of admixtures were prepared. Set 1 was prepared in 50 mL IV bags containing diluent, with final mAb1 concentration of approximately 1.6 mg/mL (FIG. 5A). Set 2 was prepared in 100 mL IV bags containing diluent, with final mAb1 concentration of approximately 0.85 mg/mL (FIG. 5B). The surface area to volume ratios were 2.7-3.9 for 50 mL IV bags and 1.7-2.5 for 100 mL IV bags. In the 50 mL IV bags, the particle distribution remained relatively constant for solutions with polysorbate 20 concentration ≥0.0004%. At concentrations below 0.0004% (w/v). At concentrations below 0.0004% (w/v), specifically 0.00034% (w/v), there is a shift towards both more and bigger particles. A similar trend was observed when the admixtures were prepared in 100 mL IV bags. Similar observations were present in IV bags for both t=0 and storage samples (FIGS. 13A-13B). Admixtures prepared with final polysorbate 20 concentration of 0.0004% (w/v) had comparatively low particle levels with a narrow distribution of particle sizes. When the polysorbate 20 concentration was reduced to 0.0003% (w/v), the particle distribution in delivered samples shifted to higher counts and larger particles. Further reducing the polysorbate 20 concentration to 0% (w/v) resulted in a dramatically shifted distribution to even larger particles and higher counts. These data suggest that particle formation and morphology are due solely to the level of polysorbate 20, and are independent of IV bag size. Particle formation and morphology are also independent of protein concentration, at least in the range tested.

FIG. 5C shows representative particle images from FI for IV admixtures containing ≥0.0004% (w/v) polysorbate 20 or ≤0.00033% (w/v) polysorbate 20. It is clear from the images that in admixtures with ≥0.0004% (w/v) polysorbate 20, the particles are small and, in some cases, may be silicone oil or air bubbles (FIG. 5C, top row). As the polysorbate concentration is reduced to ≤0.00033% (w/v) the morphology clearly shifts to larger, fibrous particles (FIG. 5C, bottom row). The particles are likely to be proteinaceous in nature as the morphology is consistent with protein aggregates. It should be emphasized that due to the mechanism of light obscuration used in analysis by LO, measuring fibrous transparent particles may not yield accurate particle concentration data, and therefore orthogonal methods such as FI are extremely useful at identifying protein instabilities which result in transparent particles.

Light obscuration is considered the gold-standard for release and stability testing with respect to particles for drug products, and USP <788> outlines criteria for acceptable levels of particles. Although no industry standard exists, flow imaging (FI) is also an important assay for characterizing particle formation in biologic drug solutions because it is generally more sensitive and can yield data on particle morphology which can help understand the nature of the particles and the degradation mechanism. These Examples demonstrate how the two techniques can complement each other. LO provided quantitative particle concentrations relative to an industry standard. FI not only provided a quantitative measurement of particles, but also showed how the nature of particle morphology changed with condition or stress. As shown, for example in FIG. 5C, there was an obvious change in particle morphology as the level of polysorbate in the IV admixture dropped below a threshold level. LO may be less sensitive to transparent or translucent particles, and cannot distinguish between intrinsic protein particles and non-protein particles that may be foreign, or result from the components of the infusion set or container. This indicates that orthogonal particle analysis methods should be employed for development studies.

TABLE 4-1
Particle concentrations measured by FI for delivered IV
admixtures that contain ≤0.0004% (w/v) polysorbate 20

| Sample | Particles/mL >10 μm | Particles/mL >25 μm |
|---|---|---|
| 0.00018% PS20 | 682 | 191 |
| 0.00025% PS20 | 695 | 154 |
| 0.00029% PS20 | 227 | 52 |
| 0.00033% PS20 | 555 | 142 |
| 0.00036% PS20 | 437 | 164 |
| 0.00039% PS20 | 760 | 160 |
| 0.00040% PS20 | 96 | 23 |

TABLE 4-2
Summary of mAb1 IV admixture quality attributes when administered using different infusion materials

| | | Material | | | | | | | |
|---|---|---|---|---|---|---|---|---|---|
| | | 100 mL PO Bag | | 100 mL PVC Bag | | | | 100 mL PO Bag | |
| | | PVC + DEHP | | PE-lined | | 100 mL PO Bag | | PE-lined | |
| | | Infusion Set | | Infusion Set | | PU Infusion Set | | Infusion Set | |
| | | 25 mL/hr | | 500 mL/hr | | 500 mL/hr | | 500 mL/hr | |
| | | infusion rate | | infusion rate | | infusion rate | | infusion rate | |
| | | | Delivered | | Delivered | | Delivered | | Delivered |
| Assay | | t = 0 | Sample | t = 0 | Sample | t = 0 | Sample | t = 0 | Sample |
| Appearance | | Pass | Pass | Pass | Pass | Pass | Pass | Pass | Pass |
| Turbidity (Increase in | | 0.00 | 0.00 | 0.00 | 0.00 | 0.00 | 0.00 | 0.00 | 0.01 |
| OD at 405 nm) | | | | | | | | | |
| Particulate | ≥10 μm | 334 | 34 | 300 | 2267 | 267 | 3234 | 500 | 600 |
| Analysis | | | | | | | | | |
| by LO | | | | | | | | | |
| (particles per | ≥25 μm | 0 | 0 | 0 | 34 | 34 | 0 | 67 | 0 |
| container) | | | | | | | | | |
| Particulate | 2-10 μm | 183 | 351 | 968 | 836 | 169 | 393 | 104 | 450 |
| Analysis | | | | | | | | | |
| by FI | ≥10 μm | 12 | 49 | 9 | 43 | 17 | 52 | 11 | 6 |
| (particles per | | | | | | | | | |
| milliliter) | ≥25 μm | 2 | 7 | 1 | 8 | 2 | 5 | 5 | 2 |
| Total Protein (mg/mL) | | 0.87 | 0.86 | 0.87 | 0.86 | 0.87 | 0.87 | 0.87 | 0.87 |
| by RP-UPLC | | | | | | | | | |
| Purity by | % HMW | 0.2 | 0.2 | 0.3 | 0.2 | 0.2 | 0.3 | 0.2 | 0.3 |
| SE-UPLC | % Native | 98.7 | 98.6 | 98.6 | 98.6 | 98.8 | 98.6 | 98.7 | 98.5 |
| | % LMW | 1.1 | 1.2 | 1.1 | 1.2 | 1.0 | 1.2 | 1.1 | 1.2 |

A sample passes appearance if the solution is essentially free from particles of foreign matter that can be observed on visual inspection. Particles by light obscuration must pass USP <788>, <6000 particles/container ≥10 μm and <600 particles/container ≥25 μm. All other assays should show no meaningful changes between t=0 and delivery through the infusion set.

TABLE 4-3
Summary of mAb1 IV admixture quality attributes when
administered using different infusion materials

| | | Material | | | | | |
|---|---|---|---|---|---|---|---|
| | | 100 mL PVC Bag | | 100 mL PO Bag | | | |
| | | PVC + DEHP | | PVC + TOTM | | 100 mL PVC Bag | |
| | | Infusion Set | | Infusion Set | | PU Infusion Set | |
| | | 500 mL/hr | | 25 mL/hr | | 25 mL/hr | |
| | | infusion rate | | infusion rate | | infusion rate | |
| | | | Delivered | | Delivered | | Delivered |
| Assay | | t = 0 | Sample | t = 0 | Sample | t = 0 | Sample |
| Color and Appearance | | Pass | Pass | Pass | Pass | Pass | Pass |
| Turbidity (Increase in | | 0.00 | 0.00 | 0.00 | 0.00 | 0.00 | 0.00 |
| OD at 405 nm) | | | | | | | |
| Particulate | ≥10 μm | 900 | 734 | 334 | 834 | 867 | 934 |
| Analysis by LO | | | | | | | |
| (particles per | ≥25 μm | 34 | 0 | 0 | 34 | 0 | 34 |
| container) | | | | | | | |
| Particulate | 2-10 μm | 1152 | 245 | 760 | 837 | 1731 | 602 |
| Analysis by FI | | | | | | | |
| (particles per | ≥10 μm | 10 | 6 | 9 | 0 | 20 | 10 |
| milliliter) | ≥25 μm | 1 | 0 | 3 | 0 | 3 | 2 |
| Total Protein (mg/mL) | | 15.2 | 15.1 | 15.2 | 15.3 | 15.1 | 15.3 |
| by RP-UPLC | | | | | | | |
| Purity by | % HMW | 0.3 | 0.3 | 0.3 | 0.3 | 0.3 | 0.3 |
| SE-UPLC | % Native | 98.9 | 99.0 | 98.9 | 98.9 | 98.9 | 98.9 |
| | % LMW | 0.8 | 0.8 | 0.8 | 0.8 | 0.8 | 0.8 |

A sample passes appearance if the solution is essentially free from particles of foreign matter that can be observed on visual inspection. Particles by light obscuration must pass USP <788>, <6000 particles/container ≥10 μm and <600 particles/container ≥25 μm. All other assays should show no meaningful changes between t=0 and delivery through the infusion set.

### Example 5: Polysorbate 20 Degradation Drug Product Shelf-Life

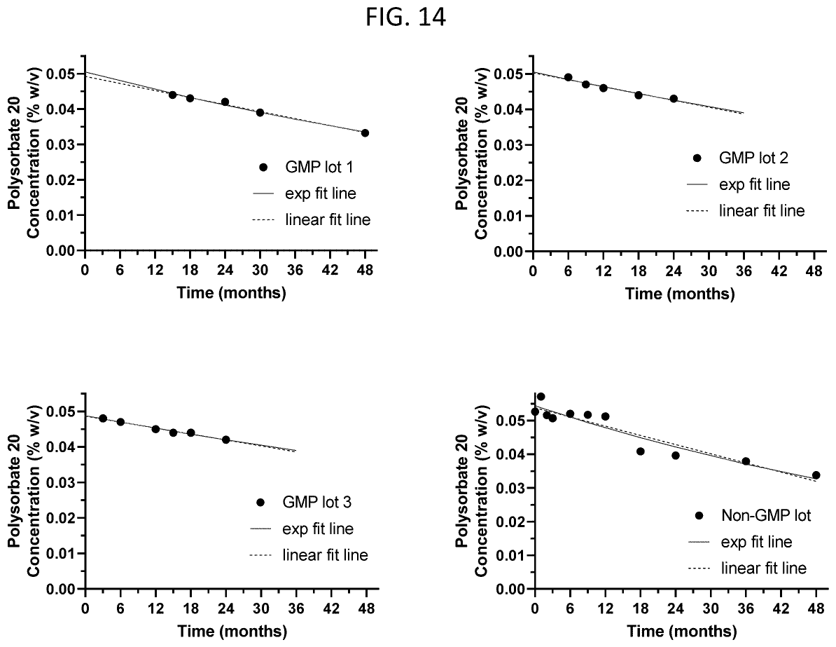

FIGS. 1C and 1F demonstrate that polysorbate was degrading over time in a DP lot manufactured from a research lot of drug substance. In order to better understand the degradation rate in the drug product, the polysorbate 20 degradation rates for three lots of drug product manufactured using Good Manufacturing Process (GMP) were measured. The degradation trend and rates were similar between the research lots and GMP lots (FIG. 14 and Table 5-3 below).

In one case, polysorbate 20 degradation was fit to a single exponential decay model to estimate the concentration of polysorbate 20 remaining at the end of shelf-life or at any time during the product shelf-life (Equation 1, above). Three DP batches from GMP manufactured lost and one lot of non-GMP DP were analyzed and used to fit the exponential decay model in Equation. 1.

Using a linear model, the average degradation rate was 0.00031% polysorbate 20 per month, in terms of the absolute amount of polysorbate in the formulation. This rate was determined using a linear fit to the measured polysorbate 20 concentration over time.

The linear fit took the form of an equation of y=mx+b, where y was the amount of polysorbate in the formulation, and x was time (in months).

The summaries of the linear fit and analysis of variants for 3 mAb1 lots are shown in Tables 5-1 and 5-2 below.

TABLE 5-1
Summary of Linear Model of Polysorbate Degradation

| | Lot 1 | Lot 2 | Lot 3 |
|---|---|---|---|
| Parameter Estimates | | | |
| Time point (m) | −.0003164 | −0.0003218 | −0.00028 |
| Standard error | 5.178e−5 | 4.326e−5 | 0.00002 |
| (month) | | | |
| t-ratio (month) | −6.11 | −7.44 | −14.00 |
| Prob > \|t\| | 0.0258* | 0.0050* | 0.0002* |
| Summary of Fit | | | |
| RSquare | 0.949153 | 0.948578 | 0.98000 |
| Rsquare Adj | 0.923729 | 0.931438 | 0.975 |
| Root Mean Square | 0.000597 | 0.000625 | 0.000346 |
| Error | | | |
| Mean of Response | 0.042 | 0.0458 | 0.045 |
| Observations (Sum | 4 | 5 | 6 |
| Wgts) | | | |

*p > 0.05

TABLE 5-2
Analysis of Variance

| | | | | |
|---|---|---|---|---|
| | Degrees | Sum of | | |
| Source | Freedom (DF) | Squares | Mean Square | F Ratio: |

Lot 1

| | | | | |
|---|---|---|---|---|
| Model | 1 | 0.00001329 | 0.000013 | 37.3333 |
| Error | 2 | 0.00000071 | 3.559e−7 | Prob > F: |
| C. Total | 3 | 0.00001400 | | 0.0258* |

Lot 2

| | | | | |
|---|---|---|---|---|
| Model | 1 | 0.00002163 | 0.000022 | F Ratio: |
| Error | 3 | 0.00000117 | 3.908e−7 | 55.3412 |
| C. Total | 4 | 0.0002280 | | Prob > F: |
| | | | | 0.050* |

Lot 3

| | | | | |
|---|---|---|---|---|
| Model | 1 | 0.00002352 | 0.000024 | F Ratio: |
| Error | 4 | 0.00000048 | 1.2e−7 | 196.0000 |
| C. Total | 5 | 0.00002400 | | Prob > F |
| | | | | 0.0002* |

*p > 0.05

TABLE 5-3
Comparison of the fit parameters for the polysorbate
20 degradation shown in FIG. 14.

| | | | | | Exponential | |
|---|---|---|---|---|---|---|
| | R² | R² | Linear | Linear | fit | Exponential |
| | exponential | linear | fit | fit | growth | fit |
| Lot | decay fit | fit | slope | intercept | rate | scale |
| GMP lot 1 | 0.9855 | 0.9901 | −0.00033 | 0.049 | −0.0085 | 0.050 |
| GMP lot 2 | 0.9559 | 0.9486 | −0.00032 | 0.050 | −0.0071 | 0.050 |
| GMP lot 3 | 0.9825 | 0.9800 | −0.00028 | 0.049 | −0.0062 | 0.049 |
| Non-GMP | 0.8921 | 0.8792 | −0.00046 | 0.054 | −0.011 | 0.054 |
| lot | | | | | | |

Using this measured degradation rate, two analyses were performed.

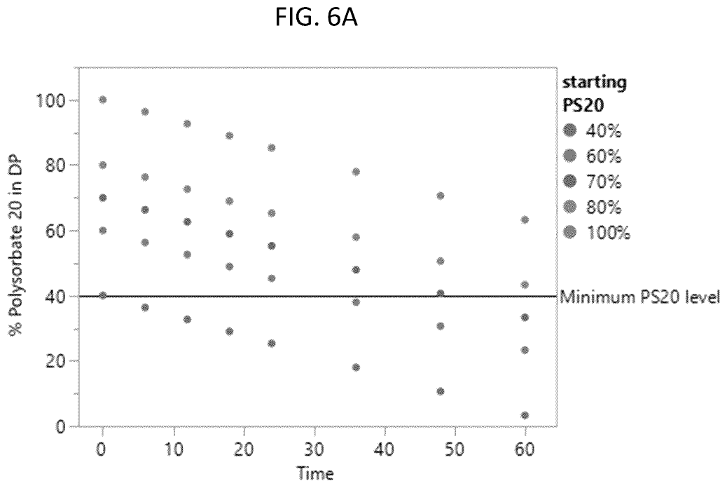

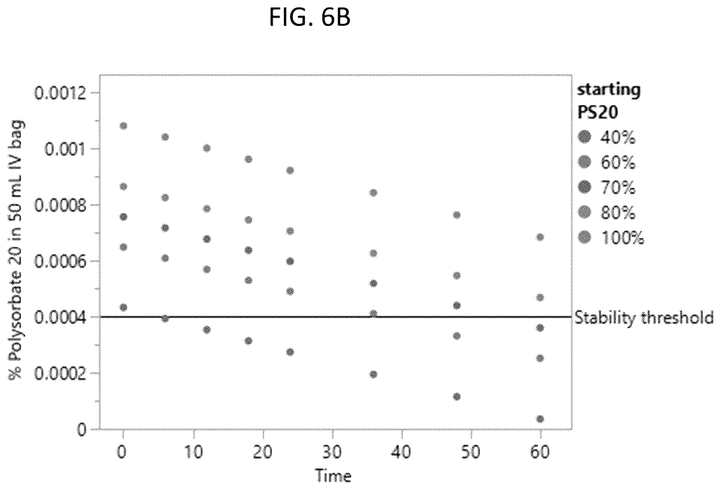

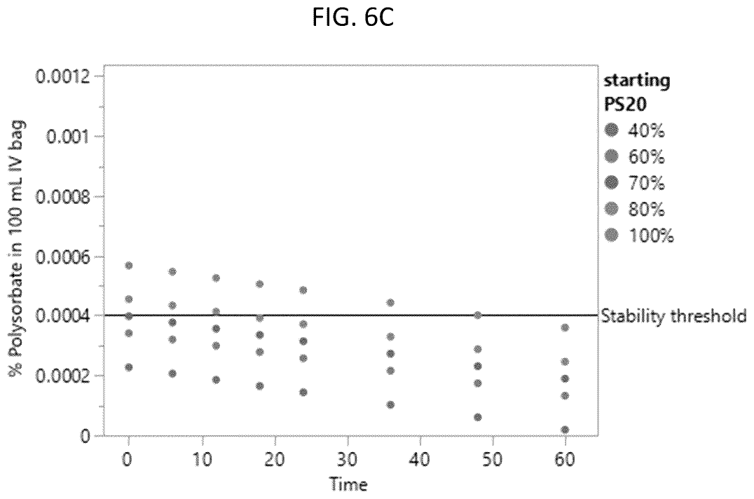

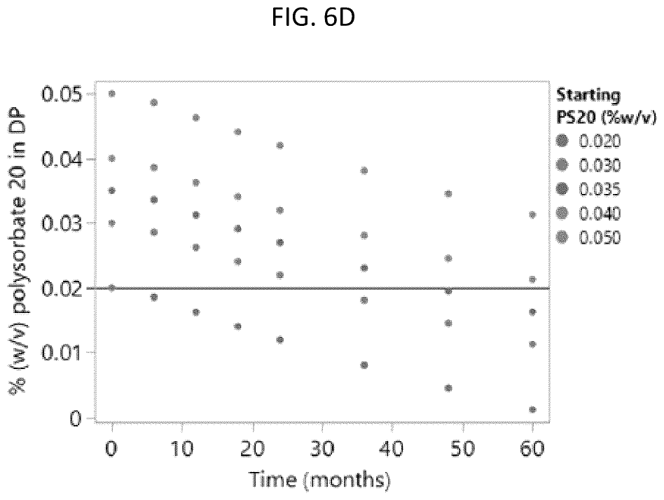

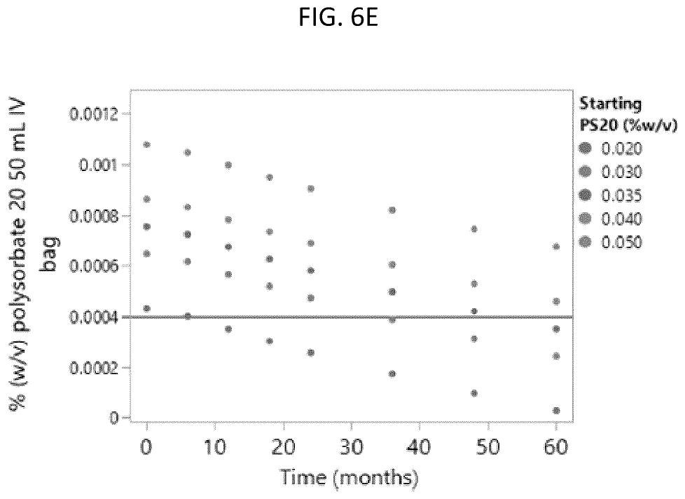

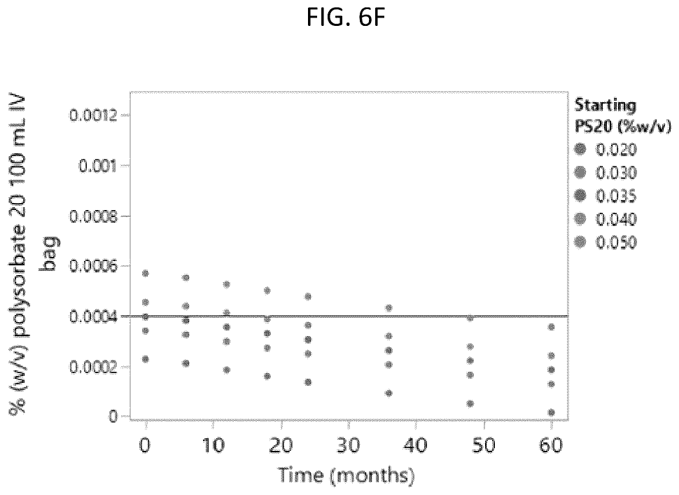

The first analysis was a model of the polysorbate 20 levels in the vialed drug product as it degrades over product shelf-life based on the starting level of polysorbate 20 (FIGS. 6A and 6C). The levels of polysorbate in available DP lots was measured, and the data was fit to a two-parameter exponential model (Equation 1, FIGS. 1C, 1F and 14) to obtain the decay rate and scale. The fitted decay rates and scales were averaged for the measured lots. These averaged parameters were used with the exponential model in Equation 1 to calculate the polysorbate concentration at any point in the DP shelf-life, up to 60 months in this case. Different starting concentrations of polysorbate 20, corresponding to the maximum manufacturing variability in DP, were used to derive several curves, each estimating what the values at different points of the shelf-life would be based on starting polysorbate concentrations. The number of stability timepoints needed to fit a reliable exponential decay and establish the degradation rate of polysorbate will vary depending on a number of factors, including when and if lipase activity is detected, the magnitude of change in polysorbate concentration, and the quality and variability of polysorbate concentration data. In an ideal case, as few as three points can give a good fit. More points may be required if the data are less than ideal. Additionally, shorter or longer times may be required depending on the same factors (i.e., a relatively large change in polysorbate concentration with a good analytical method may require 1-3 months of data with a few points for a good fit, whereas a small change measured with an assay with poorer signal to noise will require longer times and more data points). For this analysis, it was assumed that the rate of degradation of polysorbate 20 was the same regardless of the starting polysorbate 20 levels in the formulation. The rate used was measured for the formulation at the target concentration of 0.05% (w/v). Polysorbate concentration was also modelled in with formulations containing lower polysorbate concentrations. Thus, applying the degradation rate for the higher polysorbate 20 concentration to the solutions that contain lower polysorbate 20 levels represents a worst-case scenario and would conservatively overestimate the amount of degradation at the end of shelf-life. To maintain stability of the DP to mechanical or interfacial stress, and provide a safety margin, polysorbate 20 levels in the vialed DP for this mAb should be no less than 40% of the target polysorbate 20 level at the end of shelf-life, or 0.02% (w/v). This was determined during the course of surfactant screening and optimization. 0.02% (w/v) polysorbate 20 was the minimum concentration, plus a safety factor, required to see no mAb1 degradation during vigorous agitation. The model was used to predict the shelf-life of the drug product, assuming the degradation rate is the same regardless of the starting level of polysorbate 20 and that the rate is constant among lots. According to the model, the starting level of polysorbate 20 must not be less than 70% of the target polysorbate 20 concentration in order to meet the minimum 40% of target and maintain a 36-month shelf-life. To put it in terms of absolute quantity, the starting level of polysorbate 20 must not be less than 0.035% (w/v) in order to meet the minimum 0.02% (w/v) at the end of the 36 month shelf-life.

The goal of the second analysis was to understand how degradation of polysorbate 20 in the vial DP would ultimately impact the in-use stability of the IV admixture. This is important as the amount of polysorbate 20 at the end of shelf-life would be less than there would be if no degradation occurred over the course of shelf-life. This is a way to predict the starting levels in the DP are sufficient to stabilize the DP and the prepared IV admixture, and, if polysorbate 20 degradation occurs to due to the presence of lipases, if there is sufficient polysorbate 20 left at the end of shelf-life to make a stable IV admixture. Accordingly, the second analysis was a model of the polysorbate 20 concentration that would be present in IV bags if the IV admixture was prepared using drug product with the concentrations of polysorbate 20 predicted from the first model. FIG. 6B shows the predicted polysorbate 20 concentrations in IV admixtures prepared in 50 mL IV bags, assuming the polysorbate 20 in the drug product was degrading at a rate of 0.00031% per month. This was the absolute amount of degradation per month, based on the actual percentage of polysorbate in the formulation. In this case, if the polysorbate 20 concentrations in drug product are at the target concentration upon release, there will be sufficient polysorbate 20 in the IV admixture to maintain the required quality at the minimum dose level. The model also predicts that if drug product is released at 70% of the target polysorbate 20 concentration, then after 36 months there will be sufficient polysorbate 20 in the IV admixture to maintain stability. However, if drug product is released at 60% of the target concentration then there is a risk that the polysorbate 20 concentration in the IV admixture after 36 months will be at or below the threshold 0.0004% required to maintain stability, increasing the risk of higher levels of particles in the IV admixture.

FIG. 6E shows the predicted polysorbate 20 concentrations in IV admixtures prepared in 50 mL IV bags based on the predicted polysorbate 20 concentrations in the DP modelled in FIG. 6D. The predicted polysorbate concentrations modeled in FIG. 6D were used to estimate the amount of polysorbate 20 that would be found in subsequently prepared IV admixtures. The lowest intended clinical dose, which in this case equaled 75 mg (1.25 of 60 mg/mL DP) was assumed. The volume of DP (1.25 mL) multiplied by the polysorbate 20 concentration calculated for a given time point, divided by the IV bag volume yields the estimated polysorbate 20 concentration in the IV bag at any time point and for any starting concentration of polysorbate 20 (note that the IV bag volumes used for the calculation are 58 mL and 110 mL for 50 and 100 mL bags respectively. This accounts for the average overfill commonly found in commercial IV bags). In this case, if the polysorbate 20 concentrations in DP are at the 0.05% (w/v) target concentration upon release, there will be sufficient polysorbate 20 in the IV admixture to maintain the required quality at the minimum dose level. The model also predicts that if DP is released at 0.035% (w/v) polysorbate 20 concentration, then after 36 months there will be sufficient polysorbate 20 in the IV admixture to maintain in-use stability. Lot to lot and experimental variability would make this a high-risk scenario.

FIGS. 6C and 6F show the same analysis, but for IV admixtures prepared in 100 mL IV bags. In this scenario, if drug product is released at the target polysorbate 20 level, then the level of polysorbate 20 in the IV bag is predicted to be above the 0.0004% (w/v) in-use stability threshold at 36 months. However, releasing drug product with anything less than the target concentration (0.05% (w/v)) of polysorbate 20 will result in the polysorbate 20 concentration in the IV admixture being below the in-use stability threshold and a limitation on the shelf-life of the product. These models demonstrate that polysorbate 20 levels in mAb1 should be no less than 70% of the target concentration (or, 0.035% (w/v)) in DP to ensure that not only will the drug product have sufficient levels of polysorbate 20 for mAb1 stability, but will have sufficient polysorbate 20 to stabilize the IV admixture. Additionally, 50 mL IV bags should be used for dosing of this antibody. The use of larger-volume IV bags should be avoided for this case study because of the increased chance that polysorbate levels in the IV admixture will be below the threshold level required for in-use stability.

The above Examples have demonstrated a strategy for assessing the risk of particle formation in IV admixtures for biologic drugs when residual host-cell lipases are present. A challenge associated with biologic drug products purified from Chinese Hamster Ovary cells is the presence of such lipases at levels that may be difficult to detect but that are still sufficient to significantly reduce the level of polysorbate in the drug formulation over the product shelf-life. As new and more sensitive assays are developed, polysorbate degradation can be better understood, however, the testing may be challenging for routine release and stability testing. It is thus important to understand the risk that residual host-cell lipases pose not only to the quality and stability of the drug product, but just as important, the quality and in-use stability of the IV admixture. Degradation of polysorbate may be detected in the drug product if polysorbate analysis is on the release and stability panel, however, a recent survey of companies suggests that polysorbate analysis is not common in IV admixture testing. The same survey indicated that IV admixture testing is typically done in research and development laboratories, and rarely assessed in a GMP environment. It therefore falls under the umbrella of development teams to understand the implications of polysorbate degradation on the in-use stability of IV admixtures. Development groups are responsible for understanding the proper amounts of stabilizers required in the formulation and guarantee that those levels are maintained throughout the shelf-life of the drug product. Additionally, as shown in this work, the quality of the drug product may meet all quality attributes, but the extent of polysorbate degradation may be such that impacts to quality are only observed once the biologic drug is diluted in an admixture. Furthermore, degradation of polysorbate and its impact to IV admixture in-use stability should be considered early enough in development to allow for a proper specification setting discussion or adjustment of the formulation excipients to best suit the intended use of the product. A failure to identify polysorbate concentration as a critical quality attribute during development could result in incorrect specifications as well as an incomplete GMP release and stability testing strategy. For accelerated programs that might not have a batch of DP at full shelf-life, the approach outlined in this work can provide critical information required to predict safe levels of excipients.

In the study reported here, it was determined that a threshold level of 0.0004% (w/v) polysorbate 20 must be present in the IV admixture for mAb1 in order to guarantee the solution meets the quality attributes necessary to provide a safe infusion for the intended clinical doses. In order to meet this requirement, the DP must have 0.035% (w/v) polysorbate 20 at release and >0.02% (w/v) at the end of shelf-life. This threshold level of polysorbate 20 is not universal, and every antibody (or other biologic drug) will require specific testing to determine this minimum level. For example, an antibody that required 0.01% (w/v) polysorbate 20 to stabilize to agitation stress is known in the art. Similarly, a comparison of three antibodies showed that two were stabilized to agitation stress by 0.005% (w/v) polysorbate 20 but the third required higher levels such as 0.04% (w/v). A review of commercially available antibody formulations showed that the amount (and type) of surfactant greatly varied among products (from 0.04-2 mg/mL for polysorbate 20). Degradation of polysorbate (if observed) should be a considering factor in the decision on the level of polysorbate needed for the final formulation. For this study, super-refined polysorbate 20 was used. Use of super-refined polysorbate 20 is the standard practice at Regeneron due to increased quality of the reagent. However, this work may be applicable to any grade or form of polysorbate where degradation is observed on storage stability.

Light obscuration is considered the gold standard for release and stability testing with respect to particles for drug products, and USP <788> outlines criteria for acceptable levels of particles. Although no industry standard exists, flow imaging is also an important assay for characterizing particle formation in biologic drug solutions because it is generally more sensitive and can yield data on particle morphology which can help understand the nature of the particles and the degradation mechanism. The current work demonstrates how the two techniques can complement each other. LO provided quantitative particle concentrations relative to an industry standard. FI not only provided a quantitative measurement of particles, but also showed how the nature of particle morphology changed with condition or stress. In this work, there was an obvious change in particle morphology as the level of polysorbate in the IV admixture dropped below a threshold level. LO might have some shortcomings as it may be less sensitive to transparent or translucent particles and cannot distinguish between intrinsic protein particles or non-protein particles that may be foreign or result from the components of the infusion set or container. This work suggests that orthogonal particle analysis methods should be employed for development studies. This may indeed be common practice in the biopharmaceutical industry. When surveyed about practices regarding particle analysis of IV admixtures, many companies indicated that they use both LO and FI in biologic IV admixture development studies.

IV admixtures should be tested with material that is close to or at the end of proposed expiration date. This is explicitly stated in European Union guidance documents, but not something explicitly outlined by the US Food and Drug Administration. This practice may be inconsistent among pharmaceutical companies. A survey among companies indicated that less than half of the companies surveyed include aged DP in IV admixture studies. The strategy proposed here can both inform and complement studies on aged DP as we propose a method to simulate aging and the effects of degraded excipients on the quality of IV admixtures. This can be useful to set a product shelf-life or a specification on the lower limit of polysorbate needed in the DP.

The study presented describes a way that development groups can determine the minimum amount of polysorbate required to maintain the in-use stability of IV admixtures in advance of the availability of drug product close to or at the end of shelf-life. Understanding excipient degradation will be important in developing safe IV drug products.

### Example 6: Impact of Residual Host Cell Lipase Activity on a Monoclonal Antibody

The impact of residual host cell lipase activity on monoclonal antibody stability in drug product formulations was assayed over time by looking at the distribution of charge variants, purity of the antibody, and its potency in a relevant biological assay. mAb1 was stored over the shelf-life of the drug product, 36 months, at 2-8° C. Antibody drug product formulations were not formulated as IV admixtures for this Example. The results from this Example demonstrate first, that polysorbate degradation does not impact other monoclonal antibody quality attributes long term in drug product formulations, and second, that the impacts of polysorbate degradation are only seen when the monoclonal antibody drug product is formulated as an IV admixture. Consequently, one would not see the impacts of polysorbate degradation if only assaying the drug product and not the IV admixture.

### Charge Variance Analysis by Imaged Capillary Isoelectric Focusing

The level of charge variants seen in mAb1 samples was measured using an iCE3 purchased from Protein Simple (Santa Clara, CA) with a 720NZ Autosampler purchased from Alcott (Norcross, GA). Samples were diluted with water to 2 mg/mL and 40 μL were transferred to a 96-well plate. Samples were mixed with 160 μL of MasterMix containing high purity water, 3-10 Pharmalytes purchased from Cytiva (Marlborough, Massachusetts), 1% methyl cellulose, pI marker 5.12, and pI marker 9.50 purchased from Protein Simple (Santa Clara, CA). The autosampler mixed MasterMix with samples immediately before injection on a cIEF FC-coated cartridge purchased from Protein Simple (Santa Clara, CA). 100 mM sodium hydroxide and 80 mM phosphoric acid purchased from Protein Simple (Santa Clara, CA) were used to create the pH gradient along the FC cartridge. The final composition of sample after mixing was 0.4 mg/mL mAb1, 4% 3-10 Pharmalytes, 2M urea, 0.35% methyl cellulose, and 0.005% pI marker 5.12 and 9.50. Injections were converted to CDF files and analyzed in Empower 3.

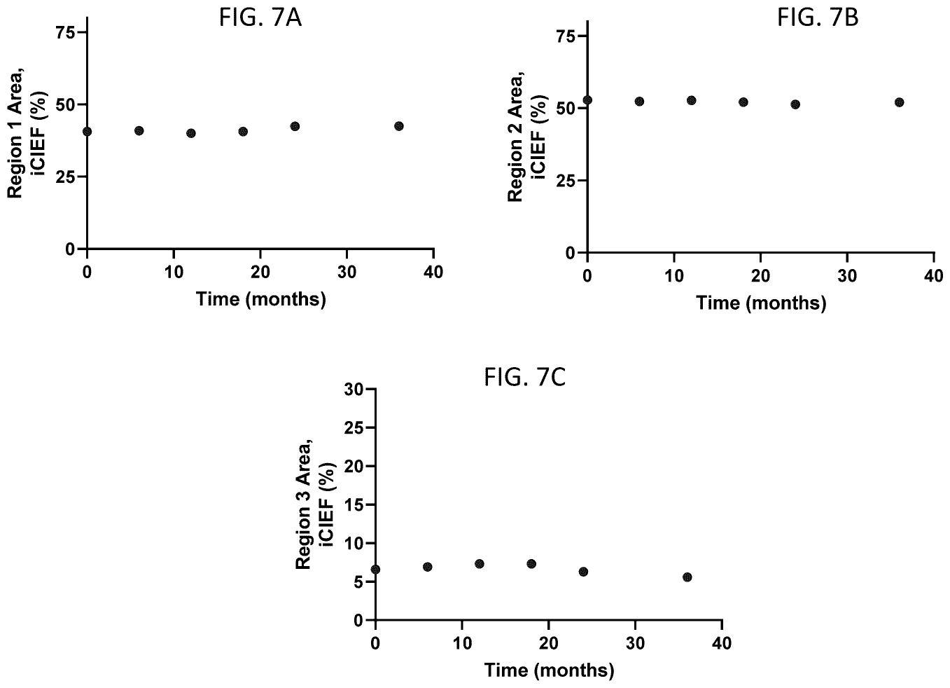

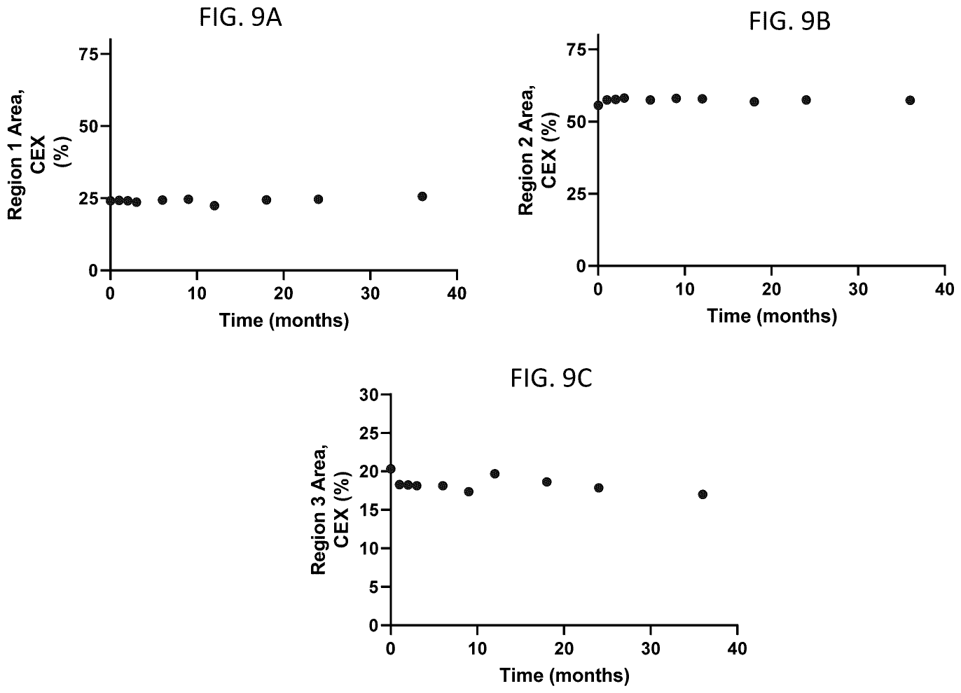

The results are shown in FIGS. 7A-7C. As can be seen from FIGS. 7A-7C, holding the samples for the indicated amount of time had little effect on the three regions analyzed by iCIEF (acidic charge variants, or Region 1 shown in FIG. 7A; main charge species, or Region 2 shown in FIG. 7B; basic charge variants, or Region 3 shown in FIG. 7C). Similar results were obtained with cation exchange chromatography (CEX, shown in FIGS. 9A-9C).

### Charge Variant Analysis by Cation Exchange-Ultra Performance Liquid Chromatography (CE-UPLC)

An orthogonal method of charge variance analysis of mAb1 formulations was determined by CE-UPLC with an Acquity UPLC H-Class system and a YMC-BioPro SP-F 5 μm, 4.6×100 mm column purchased from YMC (Devens, MA). The mobile phase consisted of 200 mM MES free acid, mobile phase B was 200 mM MES sodium salt, mobile phase C was 1 M sodium chloride and mobile phase D was high purity water. Test articles were injected along a gradient at 0.5 mL/min for 18 minutes and the absorbance of the samples measured at 280 nm. The percentage of each charge variant species was determined by the relative abundance and area under the peak.

### Purity by Size Exclusion-Ultra Performance Liquid Chromatography (SE-UPLC)

The purity of the mAb1+polysorbate 20 formulations was determined by SE-UPLC with an Acquity UPLC H-Class system and an ACQUITY UPLC BEH200 SEC 1.7 μm, 4.6×300 mm column purchased from Waters (Milford, MA). The mobile phase consisted of 10 mM sodium phosphate and 1M sodium perchlorate. Test articles (50 μg column load for both 60 and 200 mg/mL samples) were injected isocratically at 0.3 mL/min for 15 minutes and the absorbance of the samples measured at 280 nm. The percentage of each protein species was determined by the relative abundance and area under the peak.

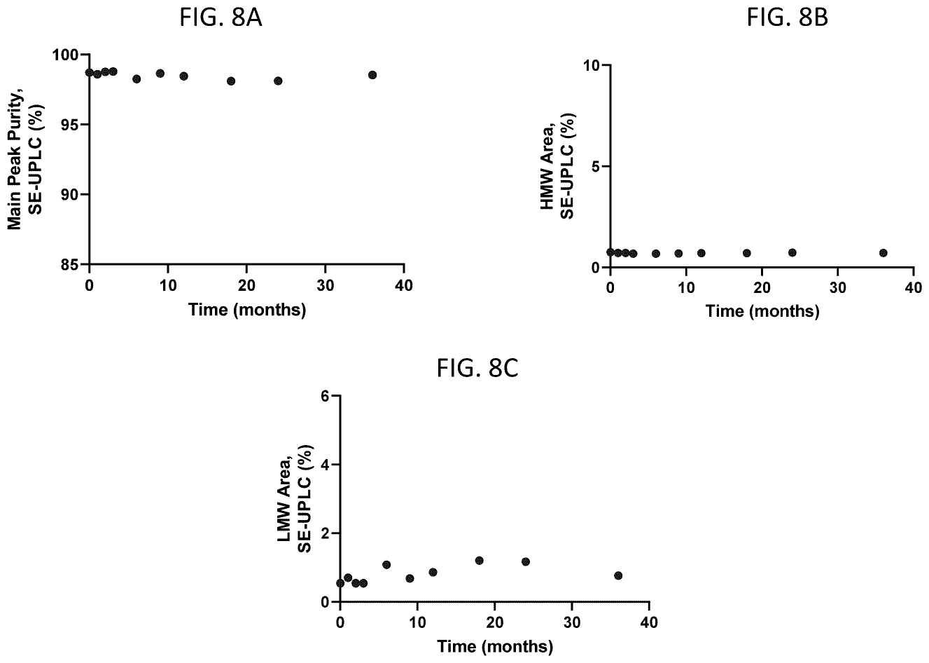

mAb1 formulations were held for the amount of time indicated in FIGS. 8A-8C, and the molecular weight species were assayed by SE-UPLC. The results are shown in FIGS. 8A-8C. Very little change in peak area over time was observed in the main peak (FIG. 8A), the high molecular weight (HMW) area (FIG. 8B), and the low molecular weight (LMW) area (FIG. 8C), indicating that molecular weight variant distribution was stable over the assayed time period.

### Purity by Microchip Capillary Electrophoresis

Purity of the DP was also assessed with non-reduced and reduced microchip capillary electrophoresis on a LabChip GXII Touch with an HT Protein Express LabChip purchased from PerkinElmer (Waltham, MA). Samples diluted to 0.5 mg/mL were mixed with lithium dodecyl sulfate buffer containing either iodoacetamide from Sigma Aldrich (St. Louis, MO) for non-reduced samples, or NuPAGE Sample Reducing Agent (10×) from Introgen (Waltham, MA) for reduced samples, and denatured at 70° C. for 10 minutes. The denatured samples were labeled with 5 μM lyophilized labeling dye from a PICO Protein Reagent Kit from PerkinElmer (Waltham, MA) and reconstituted with high purity water in a 1:1 ratio, and then incubated for 30 minutes at 35° C. Samples were quenched with a diluted stop solution containing 2.3% Stop Buffer and 16.3% Sample Buffer from a PICO Protein Reagent Kit from PerkinElmer (Waltham, MA) and high purity water, and plated on a 96 well plate from BioRad (Hercules, CA). The HT Protein Express LabChip was prepared with Protein Gel Matrix and Lower pI Marker from a PICO Protein Reagent Kit from PerkinElmer (Waltham, MA) and primed before samples were ran. Following a run, data was exported and analyzed in Empower 3.

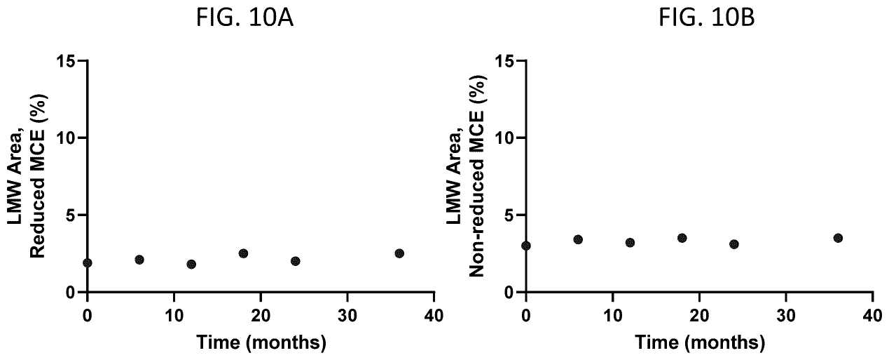

mAb1 antibody formulations were held for the amount of time indicated in FIGS. 10A-10B, and the purity of the antibody drug product was assessed by non-reduced and reduced microchip capillary electrophoresis. The results are shown in FIGS. 10A-10B. As can be seen in FIGS. 10A-10B, very little change in the LMW area was observed under either reduced or non-reduced conditions.

### Potency by Biological Assay

A cell-based luciferase reporter assay was developed using a cell line stably transfected with a luciferase reporter. The reporter cells displayed a robust luciferase expression in response to activation and this line was used for mAb1 assays.

Experimentally, A204/Smad2/3-Luc reporter cells resuspended in assay media (OptiMEM supplemented with 0.5% FBS) were plated in 96-well plates at a density of 20,000 cells per well (80 μL) and incubated overnight at 37° C. and 5% CO₂. The next day, cells were treated with 20 μL mAb1 (dilution series with concentrations ranging from 2 pM to 2 nM) and 20 μL mAb1 antigen (0.1 nM constant) and the assay plates were incubated at 37° C. and 5% CO₂ for 4 hours. Next, 120 μL ONE-Glo luciferase substrate was added to each assay plate well and luciferase activity was measured using a plate reader. The luciferase activity was recorded in relative luminescence units (RLU) and data were analyzed using the four-parameter logistic equation over an 11-point dose response curve to obtain IC50 values of mAb1. Relative potency of each test article was determined using the following formula:

Relative Potency=(IC50 Reference Standard/IC50 Test Article)*100%

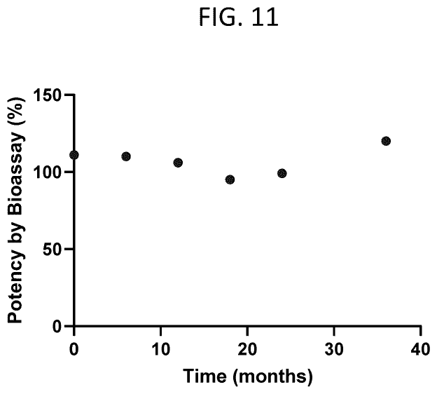

As can be seen in FIG. 11, host cell lipases had little effect on antibody potency, as measured in the bioassay.

1. A method of determining a target amount of surfactant in a liquid pharmaceutical composition comprising a protein, whereby stability of the protein is maintained in an IV admixture comprising the liquid pharmaceutical composition, comprising:
a. generating a plurality of liquid pharmaceutical compositions, wherein liquid pharmaceutical compositions in the plurality differ by an amount of the surfactant present in the liquid pharmaceutical compositions;
b. generating a plurality of IV admixtures from the plurality of liquid pharmaceutical compositions by mixing each liquid pharmaceutical composition with a diluent suitable for intravenous (IV) administration in a container;
c. simulating intravenous delivery of the plurality of IV admixtures to a subject;
d. measuring particles per container of IV admixture for IV admixtures in the plurality;
e. determining a minimum amount of surfactant whereby an amount of particles per container of IV admixture does not exceed more than 6000 particles greater than 10 μm and 600 particles greater than 25 μm; and
f. based on a shelf-life of the liquid pharmaceutical composition, the minimum amount of surfactant from step (e), and a degradation rate of the surfactant, determining the target amount of surfactant in the liquid pharmaceutical composition whereby stability of the protein is maintained in the IV admixture when the IV admixture is formulated at the end of the shelf-life of the liquid pharmaceutical composition.

2. The method of claim 1, comprising determining the degradation rate of the surfactant by:
i. determining an initial amount of surfactant in the liquid pharmaceutical composition;
ii. holding the liquid pharmaceutical composition for at least a first amount of time;
iii. determining at least a second amount of surfactant in the liquid pharmaceutical composition; and
iv. applying a model of surfactant concentration over time.

3. The method of claim 2, wherein the model comprises (a) a linear model or (b) an exponential decay model.

4. The method of 3, wherein:
(a) the linear model comprises y=mx+b, wherein y is the concentration of surfactant at time x, m is the degradation rate of the surfactant, and b is the initial amount of surfactant in the liquid pharmaceutical composition; or
(b) the exponential decay model comprises y=a×e^{bx}, wherein y is the amount of surfactant in the liquid pharmaceutical composition at time x, a is the scale, and b is the growth rate.

5. The method of claim 1, wherein simulating intravenous delivery of the plurality of IV admixtures to the subject comprises:
v. incubating the plurality IV admixtures for a first period of time at 2-8° C.;
vi. incubating for a second period of time at 21-26° C.; and
vii. pumping the plurality IV admixtures into receptacles.

6. The method of claim 5, wherein the first period of time comprises 22 to 26 hours, and wherein the second period of time comprises 6 to 10 hours.

7. (canceled)

8. The method of claim 5, wherein pumping the plurality of IV admixtures into receptacles comprises:
viii. attaching the plurality of containers to catheters and filters;
ix. holding the plurality of attached containers at about 21-26° C. for about 60 minutes;
x. connecting the plurality of containers to IV pumps; and
xi. pumping the plurality of IV admixtures through the catheters and filters into receptacles at a rate of between 25 and 100 mL/hour.

9. The method of claim 1, wherein the target amount of surfactant comprises an amount of surfactant whereby, after the liquid pharmaceutical composition has been held at storage conditions for its shelf-life, an IV admixture of the liquid pharmaceutical composition comprises less than 6000 particles greater than 10 μm and less than 600 particles greater than 25 μm per container of IV admixture.

10. The method of claim 1, wherein the target amount of surfactant is determined by a linear model and the equation y−mx, wherein y is the amount of surfactant in the liquid pharmaceutical composition at the end of shelf-life, m is the degradation rate, and x is the shelf-life.

11. The method of claim 1, wherein the surfactant comprises a non-ionic, amphoteric, cationic, or anionic surfactant.

12. (canceled)

13. The method of claim 11, wherein the non-ionic surfactant comprises a polysorbate.

14. (canceled)

15. The method of claim 13, wherein the polysorbate comprises polysorbate 20.

16. The method of claim 15, wherein the degradation rate is between 0.0001% (w/v) and 0.0005% (w/v) per month using the linear model.

17. The method of claim 15, wherein the degradation rate is 0.00031% (w/v) per month using the linear model.

18. The method of claim 1, wherein the target amount of surfactant comprises an amount of surfactant that produces an IV admixture comprising greater than or equal to 0.0004% (w/v) polysorbate 20 when the liquid pharmaceutical composition is mixed with a suitable diluent at the end of its shelf-life to produce the IV admixture, or wherein the amount of polysorbate 20 in the liquid pharmaceutical composition is greater than or equal to 0.02% (w/v) at the end of shelf-life.

19. The method of claim 1, wherein the shelf-life of the liquid pharmaceutical composition is between 6 and 60 months, inclusive of the endpoints.

20. (canceled)

21. The method of claim 1, wherein the shelf-life of the liquid pharmaceutical composition is about 6 months, 12 months, 18 months, 30 months, 36 months, 42 months, 48 months, 54 months or 60 months.

22. The method of claim 1, wherein the shelf-life comprises the maximum shelf-life wherein, when the liquid pharmaceutical composition is diluted into suitable diluent to form the IV admixture, the amount of particles per container of IV admixture does not exceed more than 6000 particles greater than 10 μm and 600 particles greater than 25 μm.

23. The method of claim 1, wherein the container comprises an IV bag.

24. The method of claim 23, wherein the IV bag is a 50 mL IV bag or a 100 mL IV bag.

25. The method of claim 1, wherein the protein comprises a therapeutic protein.

26. The method of claim 25, wherein the therapeutic protein comprises an antibody.

27. The method of claim 26, wherein the antibody comprises an antibody-drug conjugate.

28. The method of claim 25, wherein the therapeutic protein comprises a receptor Fc fusion (TRAP) protein.

29. A liquid pharmaceutical composition comprising an amount surfactant determined by the methods of claim 1, wherein the liquid pharmaceutical composition is suitable for use in an IV admixture.

30. A method of determining a maximum amount of time a liquid pharmaceutical composition comprising a protein and a surfactant can be stored (shelf-life), the method comprising:
g. generating a plurality of liquid pharmaceutical compositions, wherein liquid pharmaceutical compositions in the plurality differ by an amount of the surfactant present in the liquid pharmaceutical compositions;
h. generating a plurality of IV admixtures from the plurality of liquid pharmaceutical compositions by mixing each liquid pharmaceutical composition with a diluent suitable for intravenous (IV) administration in a container;
i. simulating intravenous delivery of the plurality of IV admixtures to a subject;
j. measuring particles per container of IV admixture for IV admixtures in the plurality;
k. determining a minimum amount of surfactant whereby an amount of particles per container of IV admixture does not exceed more than 6000 particles greater than 10 μm and 600 particles greater than 25 μm; and
l. based on a rate of degradation of the surfactant, the minimum amount of surfactant from step (e), and an initial amount of surfactant in the liquid pharmaceutical composition, determining the maximum shelf-life of the liquid pharmaceutical composition whereby stability of the protein is maintained in the IV admixture when the IV admixture is formulated at the end of the shelf-life of the liquid pharmaceutical composition.

31-57. (canceled)
